(19) United States
(12) Patent Application Publication
(10) Pub. No.: US 2026/0069159 A1
(43) Pub. Date: Mar. 12, 2026

(54) METHODS, DEVICES, AND SYSTEMS FOR MONITORING HYDRATION USING WEARABLE TECHNOLOGIES

(71) Applicant: Onda Vision Technologies, Inc., Raleigh, NC (US)

(72) Inventor: William D. Reynolds, JR., Raleigh, NC (US)

(21) Appl. No.: 19/323,121

(22) Filed: Sep. 9, 2025

Related U.S. Application Data
(60) Provisional application No. 63/693,440, filed on Sep. 11, 2024.

(51) Int. Cl. A61B 5/0537; A61B 5/00

(52) U.S. Cl. CPC A61B 5/0537; A61B 5/4875; A61B 5/6831; A61B 5/7264; A61B 5/746; A61B 2503/10; A61B 2562/166

(57) ABSTRACT

Methods, systems, and devices are disclosed herein for monitoring hydration. The hydration monitoring methods, systems and devices include a wearable device designed to capture bioimpedance data to determine a hydration status of one or more subjects. The hydration monitoring methods, systems, and devices provide continuous monitoring and analysis of hydration status of the one or more subjects and can generate alerts and recommend actions based on the determined hydration status.

### CROSS-REFERENCE TO RELATED APPLICATIONS

This application claims priority to and the benefit of U.S. Provisional Patent Application No. 63/693,440, filed on Sep. 11, 2024, the entire contents of which are herein incorporated by reference in its entirety.

### GOVERNMENT SUPPORT

This invention was made with U.S. Government support under Contract No. 1949908, a Small Business Innovative Research (SBIR) Phase I contract, awarded by the National Science Foundation (NSF); under Contract No. 2023-00570, an SBIR Phase I contract, awarded by the Department of Agriculture (USDA); and under Contract No. 2234491, an SBIR Phase II contract, awarded by the NSF. The Government may have certain rights in this invention.

### TECHNICAL FIELD

The present disclosure relates generally to health monitoring. More particularly, methods, devices, and systems are disclosed for monitoring hydration using wearable technologies.

### BACKGROUND

Hydration is essential for humans. Typically, water comprises about fifty-five percent in elderly to seventy-five percent in infants. Proper hydration is critical for reasons including regulating body temperature, delivering nutrients to cells, keeping organs functioning properly, keeping joints lubricated, and preventing infections. Additionally, proper hydration improves mood, cognition, and even sleep quality. Severe dehydration can lead to brain swelling, seizures, shock, coma, kidney failure, and death. Athletes, fishermen, agricultural workers, military service members, first responders, construction workers, and the elderly are subject to dehydration if not monitored properly. Accordingly, improved methods, devices, and systems are needed for monitoring hydration.

### SUMMARY

Methods, systems, and devices are disclosed herein for monitoring hydration. In one embodiment, a programmatic method includes (1) receiving first near real-time health data including first hydration data associated with a first subject from a first portable health monitoring device, (2) determining first analysis data based on the first near real-time health data, and (3) providing at least a portion of the first analysis data to a display. The first analysis data includes a first alert and the first alert is a notification allowing a user to administer a diagnosis of dehydration for the subject.

In some embodiments, the first hydration data may be based on bioimpedance monitoring of the first subject using the first portable health monitoring device.

In some embodiments, the first hydration data may include first water content data associated with the subject.

In some embodiments, the first hydration data may include first electrolyte data associated with the subject. In further embodiments, the first electrolyte data may include calcium data, chloride data, magnesium data, phosphorus data, potassium data, and/or sodium data.

In some embodiments, the first analysis data may include a first recommended treatment for dehydration of the first subject. In further embodiments, the first recommended treatment may include a first recommended fluid intake. In still further embodiments, the first recommended fluid intake may include a first electrolyte intake. In still further embodiments, the first electrolyte intake may include calcium, chloride, magnesium, phosphorus, potassium, and/or sodium.

In some embodiments, the first recommended fluid intake may be recommended to be administered by mouth to the subject. In other embodiments, the first recommended fluid intake may be recommended to be administered intravenously to the subject.

In some embodiments, determining first analysis data may be further based on a machine learning model. In further embodiments, the machine learning model may be based on at least one temporal convolutional network. In further embodiments, the machine learning model may be based on additional hydration data of additional subjects. In further embodiments, the additional hydration data may be recorded over at least a ten-minute period. In still further embodiments, the additional hydration data may be recorded over at least a one-hour period. In still further embodiments, the additional hydration data may be recorded over at least a twenty-four-hour minute period.

In some embodiments, the display may be embedded within the first portable health monitoring device.

In some embodiments, the programmatic method may be implemented by the first portable health monitoring device.

In some embodiments, the first portable health monitoring device may be a first portable health monitoring station. In other embodiments, the first portable health monitoring device may be a first wearable health monitoring device.

In some embodiments, the first portable health monitoring device may be a first wearable health monitoring device configured to make bioimpedance measurements on a body part (e.g., a wrist, an upper arm, a forearm, or a calf of the first subject). In further embodiments, the wearable health monitoring device may include an armband, a full-arm sleeve, a calf sleeve, or the like. In other embodiments, the wearable health monitoring device may be configured to be installed in an armband, a full-arm sleeve, a calf sleeve, and/or the like.

In some embodiments, the first wearable health monitoring device includes a wrist band with a first plurality of sensors and a first transducer. In further embodiments, the first transducer may be configured to collect the first hydration data.

In some embodiments, the display may be embedded within a mobile device. In further embodiments, the mobile device may be a laptop computer, a smart tablet, a smart phone, a smart watch, smart glasses, an extended reality (XR) headset, a virtual reality (VR) headset, an augmented reality (AR) headset, or the like. In certain embodiments, the programmatic method may be implemented by the mobile device.

In some embodiments, the display may be associated with a fixed computing device. In further embodiments, the fixed computing device may be a personal computer (PC), a workstation, a smart television (TV), a kiosk, or the like. In certain embodiments, the programmatic method may be implemented by the fixed computing device.

In some embodiments, the programmatic method may be implemented by a remote server. In further embodiments, the remote server may be a portion of a networked computing environment. In still further embodiments, the networked computing environment may be a cloud computing environment. In some embodiments, the remote server may include at least one virtualized server.

In some embodiments, the portion of the first analysis data may be provided in a format of a first dashboard to the display.

In some embodiments, the user is a non-clinical user and the first notification further allows the non-clinical user to administer the diagnosis of dehydration for the subject in absence of a medical professional.

In some embodiments, the first analysis data may further include a second alert and the second alert may be a second notification allowing the user to administer a diagnosis of hyperthermia for the subject. In further embodiments, the user may be a non-clinical user and the second notification may further allow the non-clinical user to administer the diagnosis of hyperthermia for the subject in absence of a medical professional.

In some embodiments, the first analysis data further may include a third alert and the third alert may be a third notification allowing the user to administer a diagnosis of hypothermia for the subject. In further embodiments, the user may be a non-clinical user and the third notification may further allow the non-clinical user to administer the diagnosis of hypothermia for the subject in absence of a medical professional.

In some embodiments, determining the first analysis data may be further based on data-driven signal processing using at least one of a multi-resolution technique and a multi-scale technique.

In further embodiments, determining the first analysis data may be further based on data-driven signal processing using at least one of a discrete cosine transform, a wavelet transform, an empirical mode decomposition, an empirical wavelet transform, and a variational mode decomposition.

In some embodiments, determining first analysis data may be further based on a deep learning model. In further embodiments, the deep learning model may include at least one temporal convolutional network.

In another embodiment, a computing device includes a memory, and a processor. The computing device is configured to perform a method of monitoring hydration. The method includes (1) receiving first near real-time health data including first hydration data associated with a first subject from a first portable health monitoring device, (2) determining first analysis data based on the first near real-time health data, and (3) providing at least a portion of the first analysis data to a display. The first analysis data includes a first alert and the first alert is a notification allowing a user to administer a diagnosis of dehydration for the subject.

In another embodiment, a non-transitory computer readable medium is disclosed. The computer readable medium includes a plurality of machine-readable instructions which when executed by one or more processors of a computing device are adapted to cause the computing device to perform a method of monitoring hydration. The method includes (1) receiving first near real-time health data including first hydration data associated with a first subject from a first portable health monitoring device, determining first analysis data based on the first near real-time health data, and providing at least a portion of the first analysis data to a display. The first analysis data includes a first alert and the first alert is a notification allowing a user to administer a diagnosis of dehydration for the subject.

In another embodiment, a system is disclosed for monitoring hydration in a setting. The system includes a plurality of wearable health monitoring devices associated with a plurality of subjects and a computing device wirelessly coupled with the plurality of wearable health monitoring devices. The computing device is configured for (1) receiving first near real-time health data including first hydration data from the plurality of wearable health monitoring devices, (2) determining first analysis data based on the first near real-time health data, and (3) providing a portion of the first analysis data to a display. The first analysis data includes a first alert and the first alert is a first notification allowing a user to administer a diagnosis of dehydration for at least one subject of the plurality of subjects.

In another embodiment, a programmatic method includes (1) receiving first near real-time health data including first hydration data associated with a first subject from a first portable health monitoring device, (2) determining first analysis data based on the first near real-time health data, and (3) providing at least a portion of the first analysis data to a display. The first analysis data includes a first alert and the first alert is a notification allowing a non-clinical user to administer a diagnosis of dehydration for the first subject in absence of a medical professional.

In another embodiment, a computing device includes a memory and a processor. The computing device is configured to perform a method of monitoring hydration. The method includes (1) receiving first near real-time health data including first hydration data associated with a first subject from a first portable health monitoring device, (2) determining first analysis data based on the first near real-time health data, and (3) providing at least a portion of the first analysis data to a display. The first analysis data includes a first alert and the first alert is a notification allowing a non-clinical user to administer a diagnosis of dehydration for the first subject in absence of a medical professional.

In another embodiment, a non-transitory computer readable medium is disclosed. The computer readable medium includes a plurality of machine-readable instructions which when executed by one or more processors of a computing device are adapted to cause the computing device to perform a method of monitoring hydration. The method includes (1) receiving first near real-time health data including first hydration data associated with a first subject from a first portable health monitoring device, determining first analysis data based on the first near real-time health data, and providing at least a portion of the first analysis data to a display. The first analysis data includes a first alert and the first alert is a notification allowing a non-clinical user to administer a diagnosis of dehydration for the first subject in absence of a medical professional.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The embodiments illustrated, described, and discussed herein are illustrative of the present disclosure. As these embodiments of the present disclosure are described with reference to illustrations, various modifications, or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 depicts a diagram illustrating a wearable hydration monitoring system that enables real-time monitoring of individuals to predict the onset of dehydration in accordance with embodiments of the present disclosure.

FIG. 2 depicts a diagram illustrating an Internet of Things (IoT) based system for near real-time hydration monitoring of athletes in accordance with embodiments of the present disclosure.

FIG. 3 depicts a diagram illustrating how a wearable hydration monitoring system can simultaneously monitor multiple players and multiple sports teams in accordance with embodiments of the present disclosure.

FIG. 4 depicts a diagram 400 illustrating a representative example of a team dashboard showing the relative hydration status of the team, group, or crew of individuals in accordance with embodiments of the present disclosure.

FIG. 5 depicts a diagram 500 illustrating a representative example an individual (e.g., athlete) dashboard view that shows relative hydration status in accordance with embodiments of the present disclosure.

FIG. 6 depicts a diagram 600 illustrating a representative view of an overall hydration summary dashboard of a team, group, or crew of individuals in accordance with embodiments of the present disclosure.

FIG. 7 depicts a flow diagram illustrating a data-driven framework to store, process, and analyze wearable sensor data related to hydration and other physiological areas in accordance with embodiments of the present disclosure.

FIG. 8 depicts a diagram illustrating a system platform combining wearable sensors worn by each athlete paired with cloud-based analytics that sends an alert of impending heat illness via a connected dashboard in accordance with embodiments of the present disclosure.

FIG. 9 depicts a flow diagram illustrating a workflow of an athlete-driven processing pipeline for hydration monitoring in accordance with embodiments of the present disclosure.

FIG. 10 illustrates a recording of a phase angle of a subject's bioimpedance profile during Rest, Walk, and Run of treadmill exercise using a wearable system according to at least one aspect of the present disclosure.

FIG. 11 shows a representative example of a Deep Hydration Model for bioimpedance time series prediction and analysis constructed from temporal convolutional neural networks.

FIG. 12 depicts a diagram illustrating a system for near real-time hydration monitoring to address heat-related illness in farmworkers in accordance with embodiments of the present disclosure.

FIG. 13 depicts a diagram illustrating an agricultural example of how the system of FIG. 12 transforms a standalone farmworker into the connected IoT wearable hydration monitoring platform in accordance with embodiments of the present disclosure.

FIG. 14 depicts a diagram 1400 illustrating a healthcare example of how the system of FIG. 12 transforms a standalone patient into the connected IoT wearable hydration monitoring platform.

FIG. 15 depicts a diagram 1500 illustrating an agricultural use for a wearable hydration monitoring system with farmworkers harvesting and planting different crops in accordance with embodiments of the present disclosure.

FIG. 16A illustrates a Cole spectrum plot based on data from a wearable hydration monitoring system in accordance with embodiments of the present disclosure.

FIG. 16B illustrates a Phase Angle vs Frequency plot based on data from a wearable hydration monitoring system in accordance with embodiments of the present disclosure.

FIG. 16C illustrates a resistivity versus frequency plot based on data from a wearable hydration monitoring system in accordance with embodiments of the present disclosure.

FIG. 16D illustrates a reactance versus frequency plot based on data from a wearable hydration monitoring system in accordance with embodiments of the present disclosure.

FIG. 17 depicts a diagram illustrating an agricultural use case of how a wearable hydration monitoring system may be integrated into a tractor, truck, or other vehicle for monitoring the hydration of outdoor workers (e.g., agricultural workers) in accordance with embodiments of the present disclosure.

FIG. 18 depicts a diagram illustrating an example use case of monitoring hydration of delivery and/or mail service workers in accordance with embodiments of the present disclosure.

FIG. 19 depicts a diagram illustrating how dehydration manifests as an imbalance between water-intake and water-output in accordance with embodiments of the present disclosure.

FIG. 20 depicts a diagram illustrating example of human thermoregulation showing hydration and skin blood flow as two key indicators of how the body dissipates heat during exertional heat stress in accordance with embodiments of the present disclosure.

FIG. 21 illustrates a representative example of the signal decomposition employing adaptive empirical wavelet method.

FIG. 22 depicts a diagram illustrating a wearable hydration monitoring system employing a hybrid edge-cloud architecture to deliver the real-time hydration monitoring to, for example, the athletic staff or medical staff in accordance with embodiments of the present disclosure.

FIG. 23 depicts a diagram illustrating a continuous stream of data for training and an artificial intelligence (e.g., deep learning) model deployment in accordance with embodiments of the present disclosure.

FIG. 24A illustrates a Cole model plot for wearable bioimpedance measurements using a wearable hydration monitoring system in according with embodiments of the present disclosure.

FIG. 24B illustrates a Cole model plot for wearable bioimpedance measurements using a wearable hydration monitoring system in according with embodiments of the present disclosure.

FIG. 24C illustrates a Cole model plot for wearable bioimpedance measurements using a wearable hydration monitoring system in according with embodiments of the present disclosure.

FIG. 24D illustrates a Cole model plot for wearable bioimpedance measurements using a wearable hydration monitoring system in according with embodiments of the present disclosure.

FIG. 25 illustrates a path of high and low frequency current through human tissue.

FIG. 26 depicts a diagram illustrating a wearable hydration monitoring system providing a framework for inter-athlete transfer learning approach for large-scale model (e.g., deep learning) deployment in accordance with embodiments of the present disclosure.

FIG. 27 depicts a graph illustrating an example of internal temperature versus skin blood flow (SkBF) describing athlete-specific thermoregulation sensitivity to exertional heat stress in accordance with embodiments of the present disclosure.

FIG. 28 depicts a diagram illustrating a framework for bioimpedance denoising using a data-driven feedback engine in accordance with embodiments of the present disclosure.

FIG. 29 depicts a block diagram of a hydration monitoring system according to embodiments of the present disclosure.

FIG. 30 depicts a block diagram of a hydration monitoring system according to embodiments of the present disclosure.

FIG. 31 depicts a block diagram of a hydration monitoring system according to embodiments of the present disclosure.

FIG. 32 depicts a block diagram of a hydration monitoring system according to embodiments of the present disclosure.

FIG. 33 depicts a block diagram of a hydration monitoring system according to embodiments of the present disclosure.

FIG. 34 depicts a block diagram of a hydration monitoring system according to embodiments of the present disclosure.

FIG. 35 depicts a wearable device of a hydration monitoring system according to embodiments of the present disclosure.

FIG. 36 depicts a diagram illustrating the wearable device and wearable armband of FIG. 35 in accordance with embodiments of the present disclosure.

FIG. 37 illustrates an exploded view of a hydration monitoring wearable device 3600 according to at least one embodiment of the present disclosure.

FIG. 38A illustrates an electrode configuration of a hydration monitoring wearable device according to at least one embodiment of the present disclosure.

FIG. 38B illustrates an electrode configuration of a hydration monitoring wearable device according to at least one embodiment of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Methods, systems, and devices are disclosed herein for monitoring hydration using wearable technologies. Further disclosed herein is a hydration monitoring system which represents a paradigm shift from reaction-based methods to a proactive-based approach to predict the onset of dehydration that may occur through exertional heat illness, medical conditions, occupation or mission. Additionally, abrupt changes in hydration levels induced by exertion, medical conditions, occupation, or mission can be detected.

System real-time hydration monitoring fills an opportunity gap by providing next-generation athletic safety and performance. Proper hydration status reduces fatigue, muscle strains, and core body temperature while maintaining mental awareness. The system is applicable for all major outdoor sports where hydration and heat exposure represent serious health risks. A wearable system sends alert messages to the athletic trainers and/or coaches before an athlete's hydration status decreases to dangerous and/or life-threatening levels. The wearable system transmits sensor data to the cloud for data storage and processing. Algorithms monitor and predict changes in the athlete's hydration level. If a dehydration event is detected, then alert messages are sent back to the athletic trainers indicating which athlete is at risk. As a result, athletic trainers benefit from immediately knowing which athlete needs to rest and rehydrate.

The system is applicable to many use cases and market segments. The system serves people who experience abrupt changes in their hydration levels induced by athletics, exertion, occupation, or medical conditions. The solution provides early alerts and continuous monitoring to prevent adverse health outcomes.

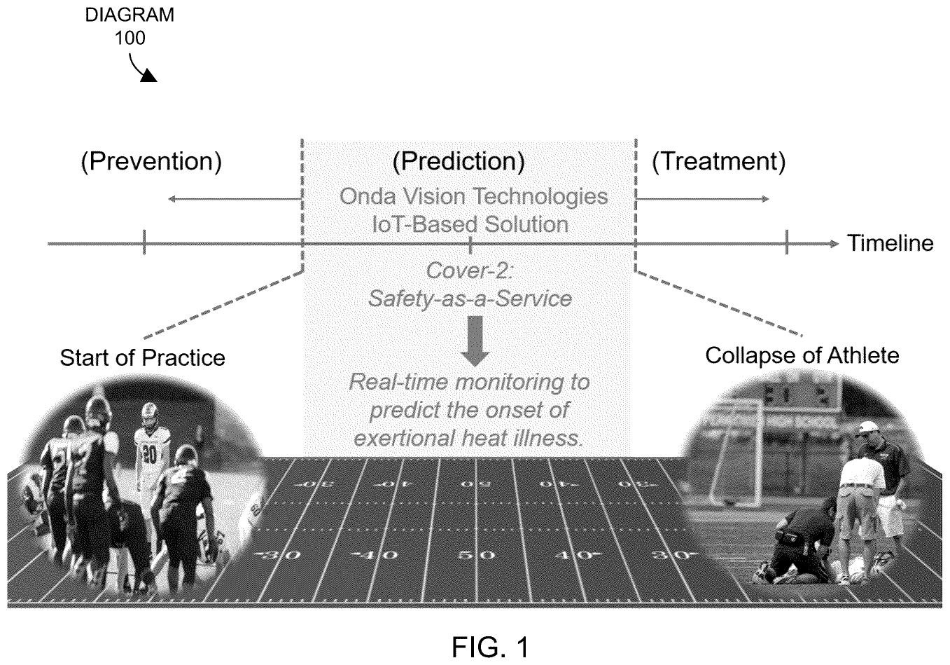

FIG. 1 depicts a diagram 100 illustrating a wearable hydration monitoring system that enables real-time monitoring of individuals to predict the onset of dehydration in accordance with embodiments of the present disclosure. The system represents a paradigm shift from reaction-based methods to a proactive-based solution that predicts unsafe health related conditions before an individual collapses whether during an athletic event, while experiencing a medical condition, while in a work environment, or on a military related mission. The system delivers personalized safety enabling, for example, an athletic trainer, coach or medical professionals to customize an individuals's activity, rest periods, and hydration patterns. The system is applicable to many areas of society grappling with safety needs against exertional heat illness, medically related dehydration, environmentally induced dehydration and/or abrupt dehydration. For example, high-risk groups such as agricultural workers, construction workers, and military personnel experience similar cases of heat illness compared with athletes. Heat-related illness propagates through both rural and urban communities. Globally, various countries and populations have experienced mass heat-related deaths in regions that are not accustomed to warm temperatures.

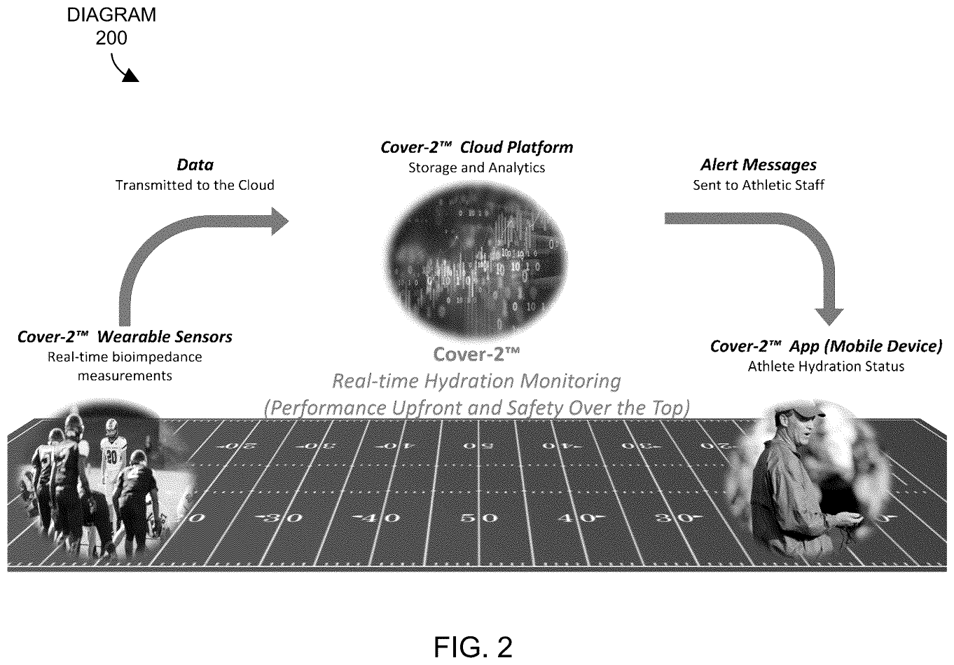

FIG. 2 depicts a diagram 200 illustrating an Internet of Things (IoT) based system for near real-time hydration monitoring of athletes in accordance with embodiments of the present disclosure. The system delivers the athlete's relative hydration changes to the athletic staff in near real-time via a smart device (e.g., smart phone, smart tablet, gateway, etc.). By using IoT, the system transforms standalone athletes into connected players providing a continuous data source about their changing hydration levels.

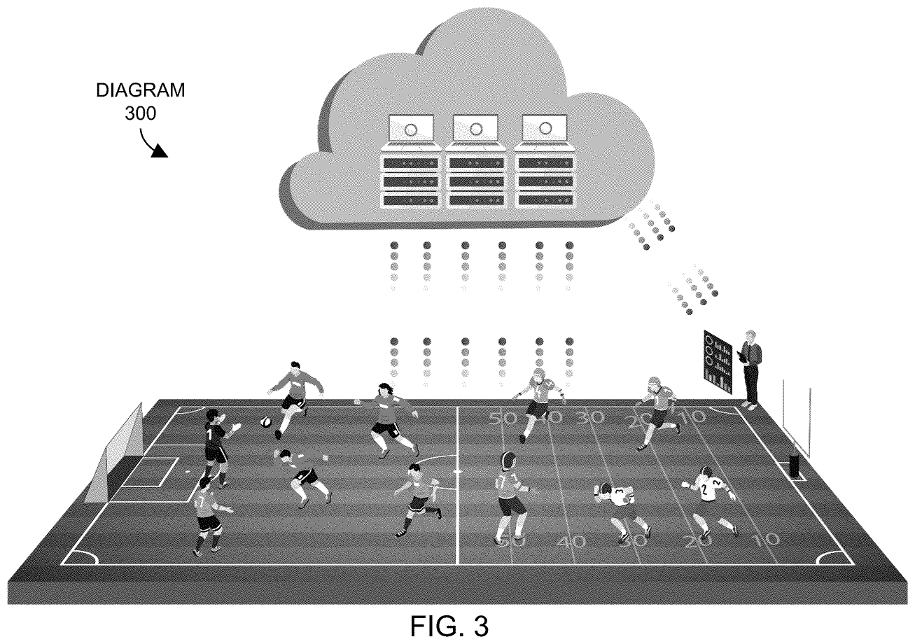

FIG. 3 depicts a diagram 300 illustrating how a wearable hydration monitoring system can simultaneously monitor multiple players and multiple sports teams in accordance with embodiments of the present disclosure. The system represents a shift in athletic care from manual field-based methods to a digital, connected, near real-time user experience linking athletic trainers with their athletes. The wearable system enables personalized safety, enabling athletic trainers to customize player activity, manage rest periods, and adjust athlete hydration behaviors.

Current acclimatization methods operate on a broad scale assuming all individuals react the same to thermal stress. However, individual responses to environmental conditions are known to vary widely. The system operates on a granular level treating each person individually to provide customized safety, for example, during exercise in hot and/or humid conditions. A key discriminator of the system comprises a heat profile tailored to the unique physiological characteristics of each individual for personalized safety. Exertional heat stroke can cause death or permanent organ damage if the duration of hyperthermia exceeds the ability of the body to tolerate the thermal stress. This duration is typically a thirty-to-sixty-minute time window. Additionally, the system is well suited to detect dehydration associated with hypothermia.

Human thermoregulation is a complex system of integrated mechanisms designed to maintain body temperature in a narrow range. Individuals who are unable to thermoregulate during exercise, who are experiencing a medical condition, or engaged in heated working environments are vulnerable to various forms of heat-related illnesses. The level of severity dramatically depends on the individual's physiological characteristics and sensitivity to heat. For example, an athlete's working muscles produce a considerable amount of heat during exercise. Heat production may be fifteen to twenty times higher during exercise than when at rest. This situation can raise core body temperature one degree Celsius every five minutes in the absence of thermoregulatory adjustment. The generated heat, in addition to the ambient heat from the environment, must be offset by the body's thermoregulation system for heat dissipation to avoid hyperthermia. In hot and humid conditions, evaporative cooling is the most effective method of transporting heat from the core to the skin surface via the blood.

Dehydration poses a significant risk factor for hyperthermia during excessive demands for heat dissipation. Monitoring a person's water content is crucial to the body as it regulates critical body functions including body temperature, blood pressure, and heart rate. Near real-time tracking of hydration level is especially beneficial to people who live or work in extreme conditions, such as soldiers and first responders. Dehydration also decreases athletic or work performances inviting other types of injuries. Moreover, dehydration poses severe risks during physical training of young athletes.

Hydration of the body is an essential physiological parameter to measure but is challenging to measure accurately. Techniques for assessing dehydration include observing a person's appearance (e.g., sunken eyes, cracked lips, etc.), or by examining the volume and color of urine. More quantitative assessment relies on measuring the change in hydration by weighing a naked person before and after exercise or by measuring electrical properties change. Most hydration monitors on the market evaluate the hydration level by measuring physical properties such as conductance, capacitance, and impedance. For reliable readings, the planar electrodes require manual placement against the skin. Shortcomings related to these devices include personal discomfort and large form factors.

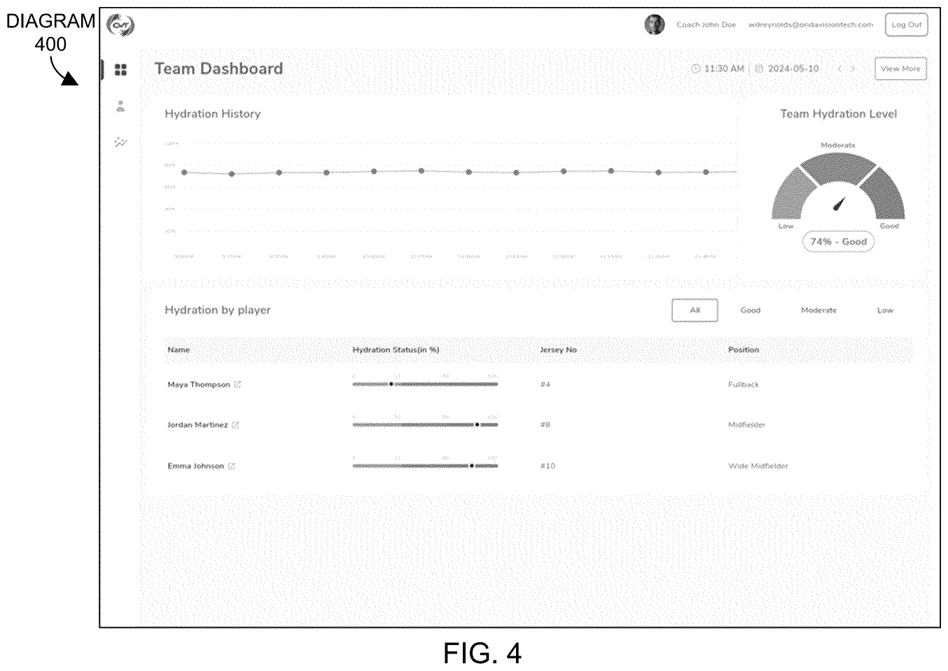

FIG. 4 depicts a diagram 400 illustrating a representative example of a team dashboard showing the relative hydration status of the team, group, or crew of individuals in accordance with embodiments of the present disclosure. The dashboard displays the person's hydration levels through color-coded features and/or shaded bars/icons. For example, red for low hydration, yellow for moderate hydration, and green good hydration may be used as indicators. Numerical values indicate the relative hydration status (e.g., percentage) measured from their baseline.

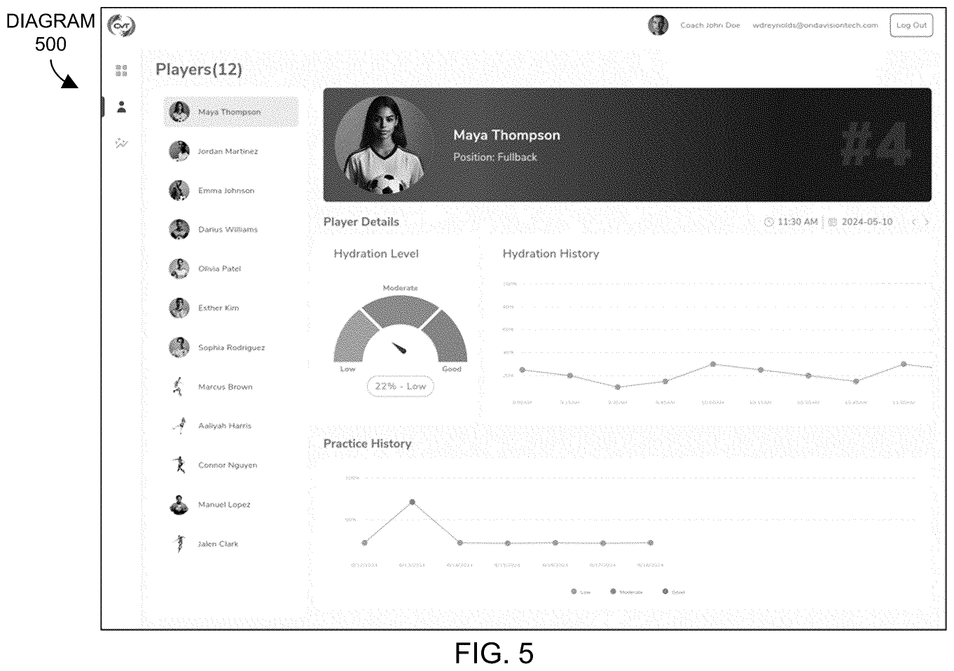

FIG. 5 depicts a diagram 500 illustrating a representative example an individual (e.g., athlete) dashboard view that shows relative hydration status in accordance with embodiments of the present disclosure. This individual dashboard also shows a historical or temporal (e.g., longitudinal) view of each individual person. Features include showing unique wearable sensor identification number, the person's name, sport, position, and color-coded and/or shaded hydration status for easy reference, quick decision-making, and personal management. The numerical values and chart indicate the relative hydration status measured from their baseline over a defined timeline (e.g., hours, days, weeks, months, year).

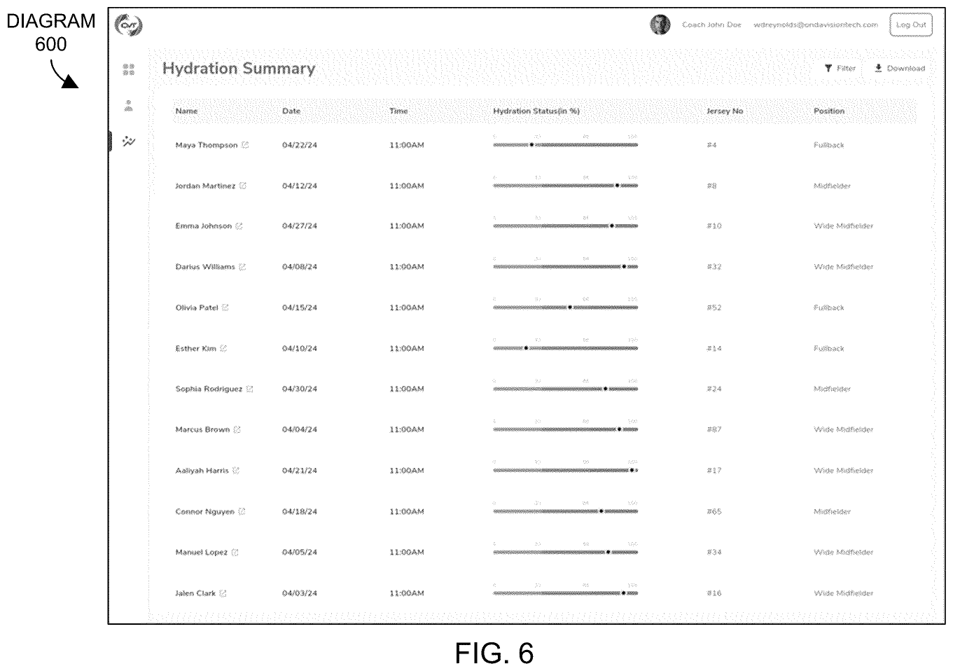

FIG. 6 depicts a diagram 600 illustrating a representative view of an overall hydration summary dashboard of a team, group, or crew of individuals in accordance with embodiments of the present disclosure. The players' hydration levels are indicated through color-coded and/or shaded bars/icons. The summary data may be organized to list the individuals in order of their hydration status. For example, the individuals are ranked based on the numerical values indicating their relative hydration status (e.g., percentage) measured from their baseline.

The wearable system exploits the natural electrical response of the body to the changes in water content. Current methods for assessing hydration status rely on blood biochemistry including plasma osmolality and electrolytes, urine tests, and weight changes. However, blood sample collection is invasive, and time-consuming laboratory analysis often delays treatment by hours. Under extreme heat conditions, dehydration reduces human thermoregulation causing death or permanent organ damage when the duration of hyperthermia exceeds the ability of the body to tolerate the thermal stress. A key discriminator of the systems disclosed herein comprises a heat profile tailored to the unique physiological characteristics of each individual for personalized safety. The heat profile stems for monitoring and detecting changes in the athlete's hydration status. Current acclimatization methods operate on a broad scale assuming all athletes react the same to thermal stress. However, individual responses vary widely to environmental conditions.

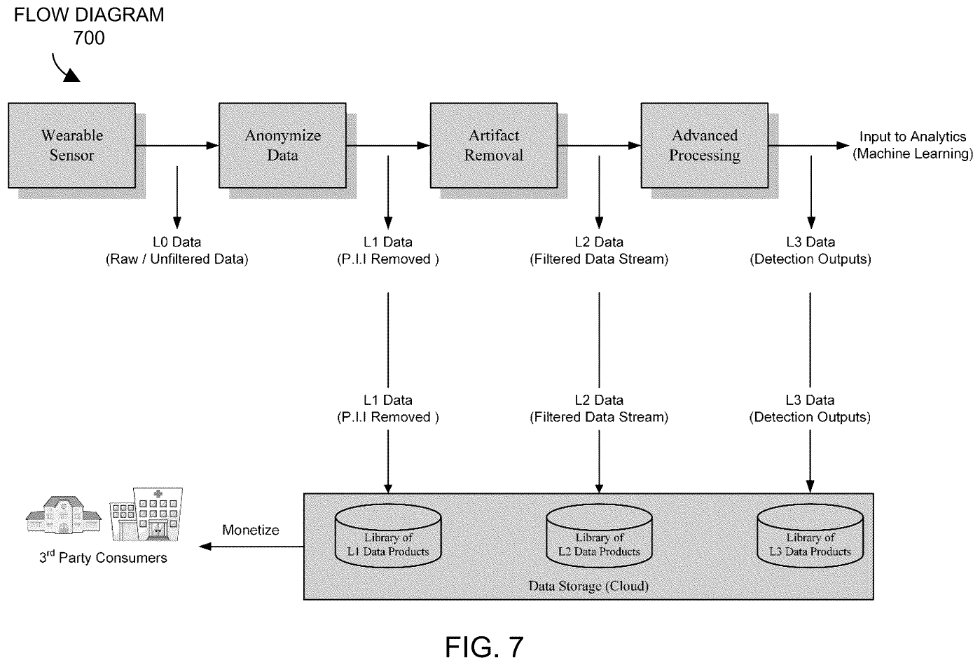

FIG. 7 depicts a flow diagram 700 illustrating a data-driven framework to store, process, and analyze wearable sensor data related to hydration and other physiological areas in accordance with embodiments of the present disclosure. The data-driven framework is configured for developing a machine learning model for hydration analysis. The system collects wearable sensor data generating the raw or unfiltered data. This step creates Level 0 data that can be stored for future development and analysis. The next step anonymizes the data by removing any personal identifiable information. The step creates Level 1 data that can stored for development and analysis. The next step filters the anonymized data using various signal processing techniques (e.g., wavelet analysis, Fourier analysis) to remove unwanted noise or spurious data points. This creates Level 2 data that can be stored for further processing and analysis. The final step applies advanced processing to detect physiological events (e.g., hydration changes) and outputs a set of detection features. This creates Level 3 data that can stored for future development or passed along to machine learning data analytics. The Level 1, Level 2, and Level 3 data sets generates a library of data products.

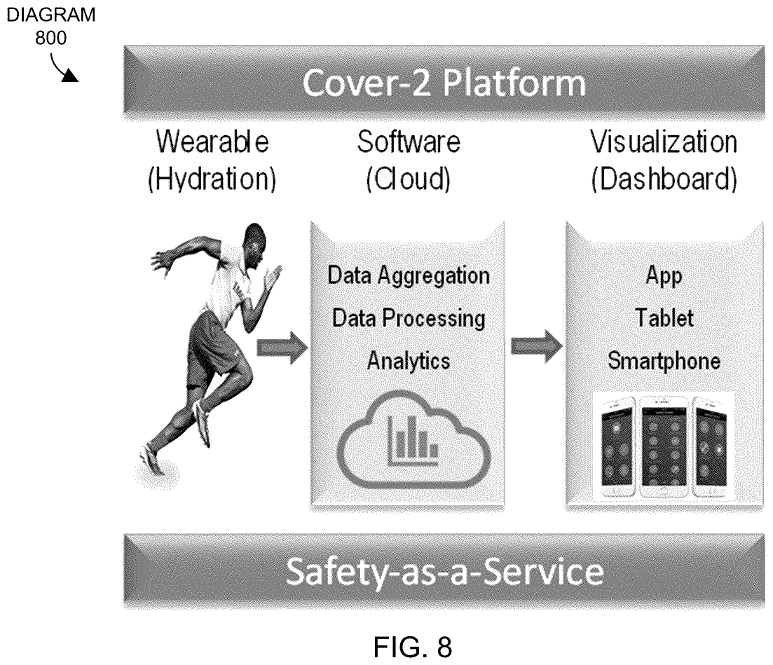

FIG. 8 depicts a diagram 800 illustrating a system platform combining wearable sensors worn by each athlete paired with cloud-based analytics that sends an alert of impending heat illness via a connected dashboard in accordance with embodiments of the present disclosure. The system provides safety-as-a service for preventing dehydration.

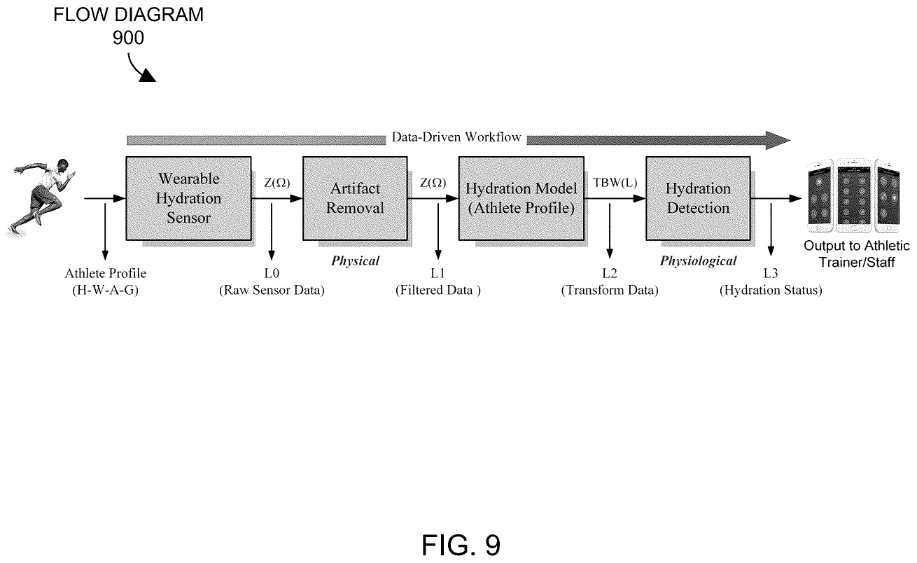

FIG. 9 depicts a flow diagram 900 illustrating a workflow of an athlete-driven processing pipeline for hydration monitoring in accordance with embodiments of the present disclosure. The workflow adapts to the athlete's unique physiological characteristics driven by wearable bioimpedance sensor data. A raw bioimpedance sensor data feeds into a hydration analysis module for transforming the data into intrinsic mode functions. A hydration filtering module removes unwanted motion artifacts and maintains the relevant modes to drive the deep learning modules. For example, the hydration analysis and hydration filtering modules define a framework for identifying and removing unwanted oscillations (e.g., walk, run, etc.) from the input signal. The filtered bioimpedance data serves as input data to the hydration monitoring module to generate the deep learning prediction model. The output denotes the athlete's predicted hydration status delivered to the athletic trainer/staff via a mobile device (e.g., smart phone, smart tablet, gateway, etc.).

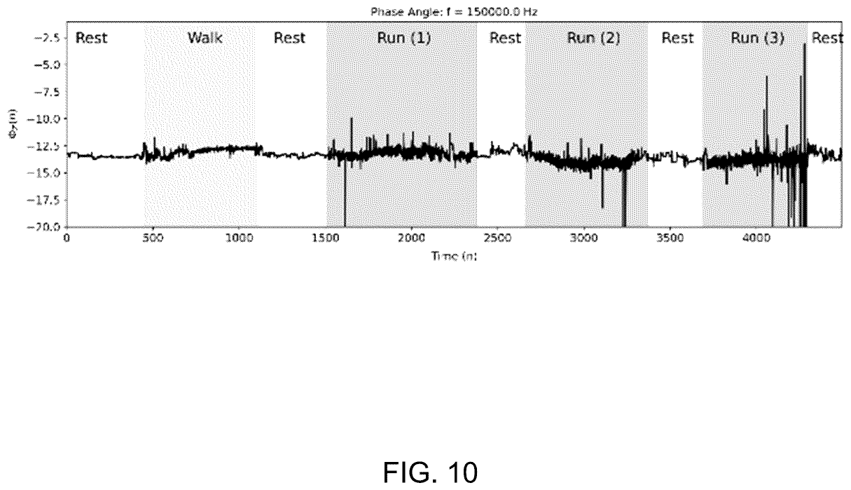

FIG. 10 illustrates a recording of a phase angle of a subject's bioimpedance profile during Rest, Walk, and Run of treadmill exercise using a wearable system according to at least one aspect of the present disclosure.

Overall, these modules improve signal integrity prior to passing the data along to the deep learning module. The wearable system develops predictive analytics based on the bioimpedance profile of the athlete. Advancements in artificial intelligence and deep learning (e.g., temporal convolutional networks) are employed to predict and monitor hydration changes over time. The innovative monitoring approach offers practical advantages including continuous data streams from our wearable sensor provide data samples for training, testing, and validating the deep learning models. The system learns the athlete's unique response as more data becomes available through repeated use of the wearable device.

The wearable system exploits the unique capability of the wearable device to monitor bioimpedance measurements from the extracellular and intracellular fluid compartments. Continuous measurements generate a bioimpedance profile unique to each athlete that captures the fluid dynamics during dehydration events. The system can formulate hydration monitoring as a time series prediction problem, where the signal X={x⁽¹⁾, x⁽²⁾, . . . , x⁽ⁿ⁾} represents bioimpedance measurements as a function of time (t). The model learns to predict the next value, x⁽ⁿ⁺¹⁾, from the previous measurements X:

$$p ( {x}^{( n + 1 )} \vert {x}^{( n )} , {x}^{( n - 1 )} , … {x}^{( 1 )} )$$

here the model represents a causal prediction problem depending only on measurements up to time t=n. Therefore, the model prediction does not depend on future bioimpedance measurements.

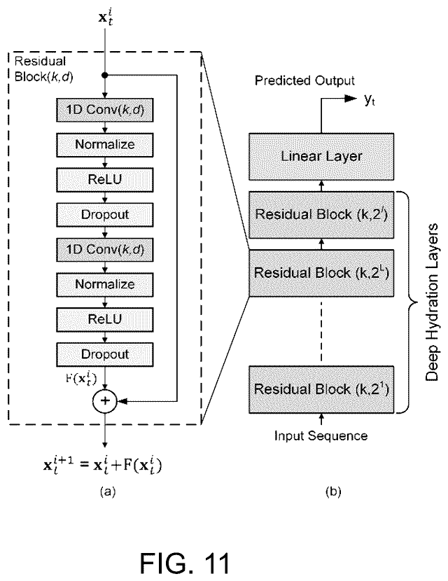

FIG. 11 shows a representative example of a Deep Hydration Model for bioimpedance time series prediction and analysis constructed from temporal convolutional neural networks. To construct the Deep Hydration Model, multiple residual blocks are stacked where the output of the previous block serves as input to the next block. The Deep Hydration Model captures the temporal patterns of the bioimpedance profile at different time scales ensuring prediction over very long signal histories. The model lays the foundation for developing data-driven workflows for athlete hydration monitoring. Through repeated use of the wearable device and as more data become available, the accuracy of the results is improved.

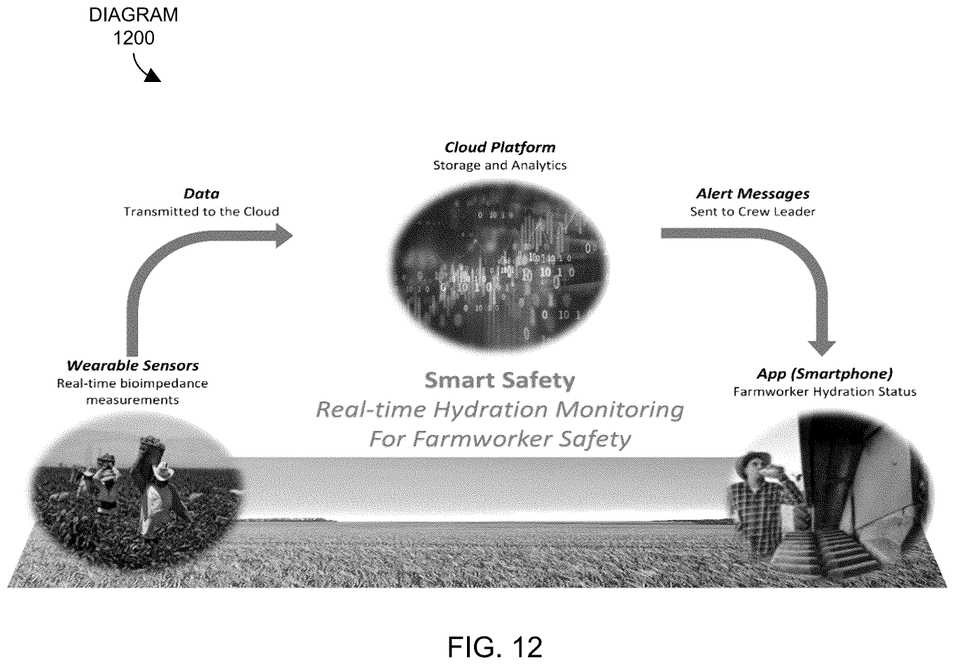

FIG. 12 depicts a diagram 1200 illustrating a system for near real-time hydration monitoring to address heat-related illness in farmworkers in accordance with embodiments of the present disclosure. Advantageously, the hydration monitoring platform enhances the safety performance of agricultural operations and overcomes workplace barriers that limit the adequacy of farmworkers' fluid intake strategies. The hydration monitoring platform is configured to support farm operational management for a plurality of workers and/or farms.

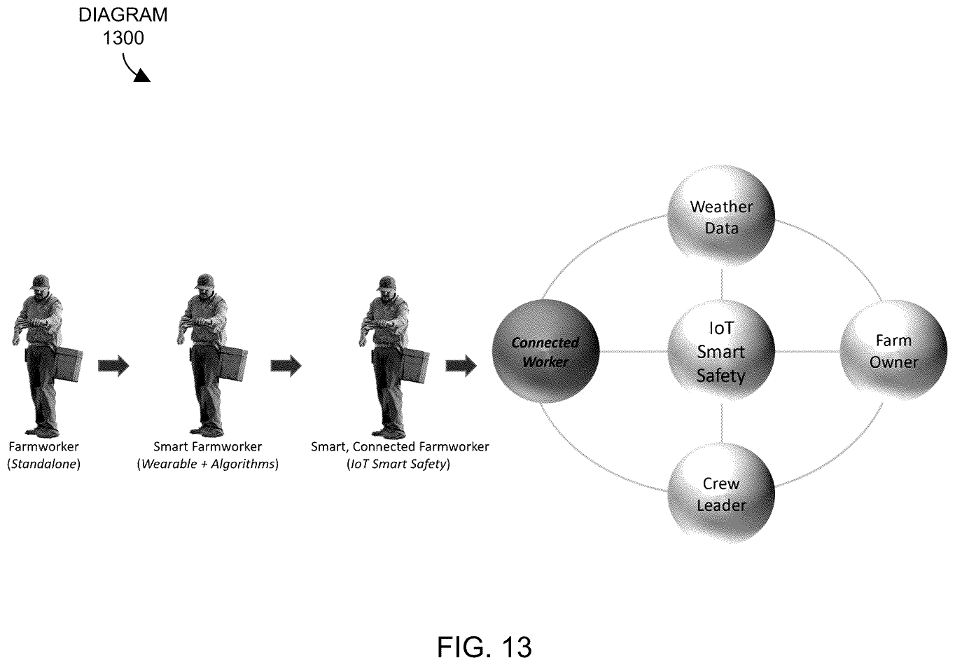

FIG. 13 depicts a diagram 1300 illustrating an agricultural example of how the system of FIG. 12 transforms a standalone farmworker into the connected IoT wearable hydration monitoring platform in accordance with embodiments of the present disclosure. The system shifts the focus from the individual to the group or crew of farmworkers to operate under these constraints. The IT infrastructure enables large-scale monitoring with workers distributed across a broader spatial coverage than commercially available devices. The IoT platform supports large-scale deployment and links the farmworker to an array of stakeholders. Workers or third-party persons can monitor the sensor data. For example, agricultural safety managers in crop production could access the data for their crew.

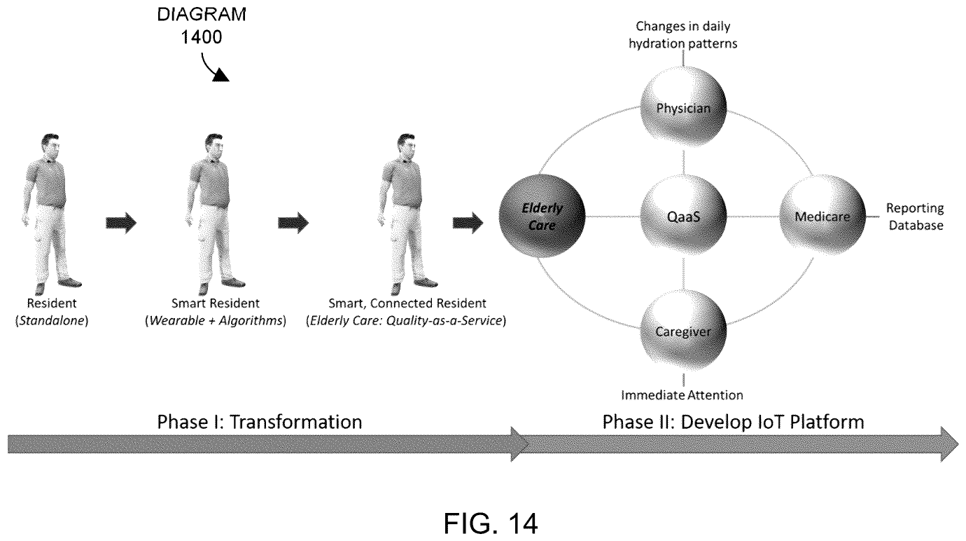

FIG. 14 depicts a diagram 1400 illustrating a healthcare example of how the system of FIG. 12 transforms a standalone patient into the connected IoT wearable hydration monitoring platform. The system enables monitoring and predicting the onset of dehydration in elderly residents living in long-term nursing facilities. For example, and without limitation, the wearable system is configured to generate an alert or notification based on a patient's hydration status. Advantageously, this enables caregivers to provide proactive medical attention for one or more patients

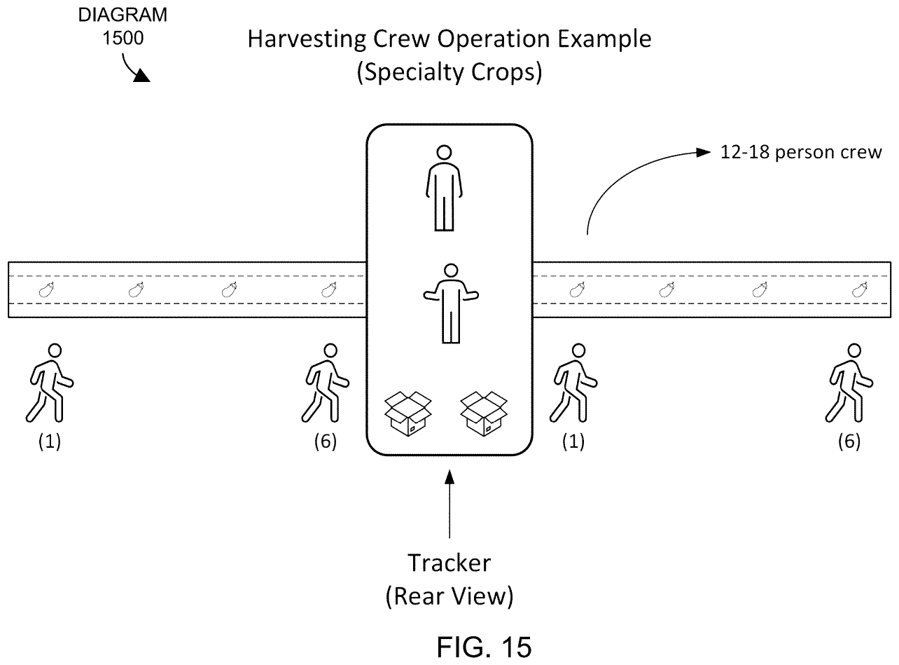

FIG. 15 depicts a diagram 1500 illustrating an agricultural use for a wearable hydration monitoring system with farmworkers harvesting and planting different crops in accordance with embodiments of the present disclosure. The system may be integrated into the tracker or vehicle to monitor the farmworkers.

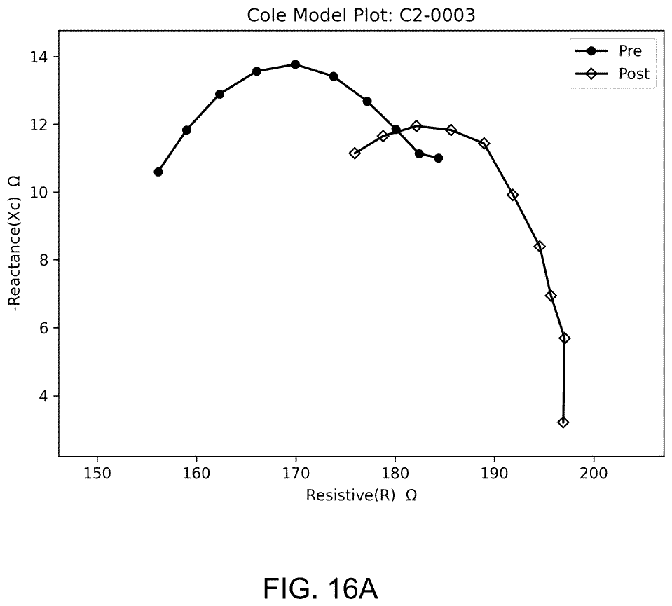

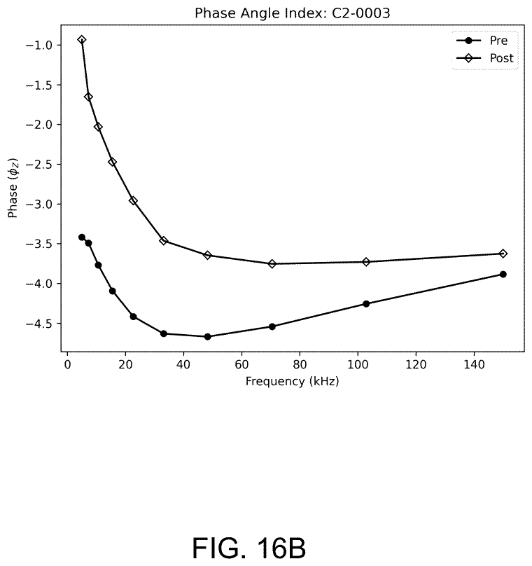

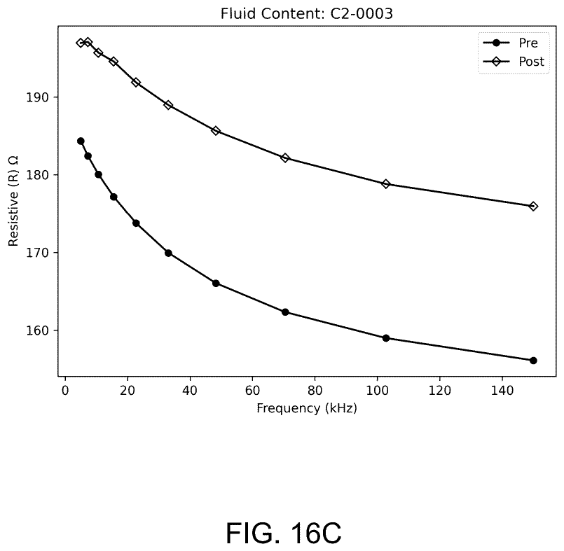

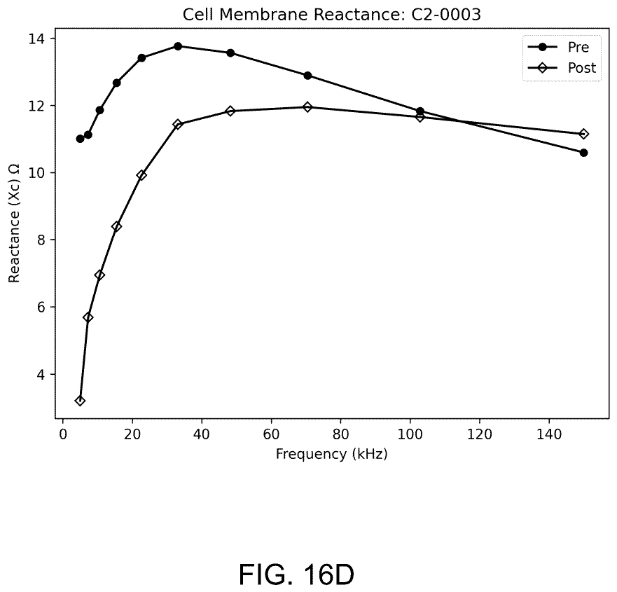

FIGS. 16A-16D illustrate representative examples of wearable bioimpedance measurements from a farmwork during harvesting. The wearable hydration monitoring system is configured for continuous monitoring of the hydration status of farm workers. FIG. 16A illustrates a Cole spectrum plot. FIG. 16B illustrates a Phase Angle vs Frequency plot. FIG. 16C illustrates a resistivity versus frequency plot. FIG. 16D illustrates a reactance versus frequency plot. These graphical representations demonstrate the collection of data from a wearable bioimpedance sensor in a rugged agricultural setting. In these examples, the wearable sensor was outfitted to a participant's upper bicep (e.g. left arm) to collect local bioimpedance measurements.

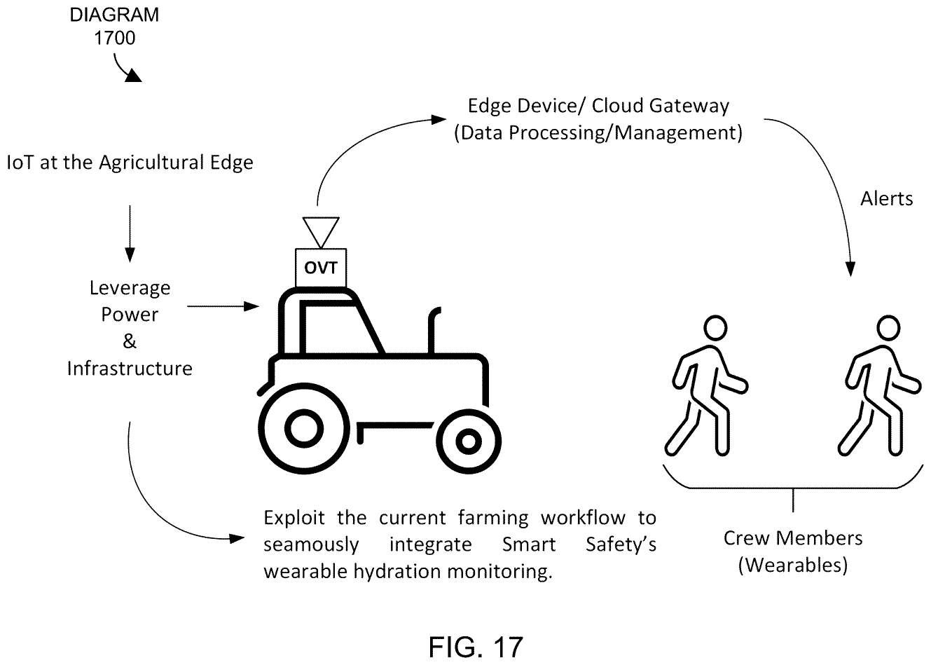

FIG. 17 depicts a diagram 1700 illustrating an agricultural use case of how a wearable hydration monitoring system may be integrated into a tractor, truck, or other vehicle for monitoring the hydration of outdoor workers (e.g., agricultural workers) in accordance with embodiments of the present disclosure.

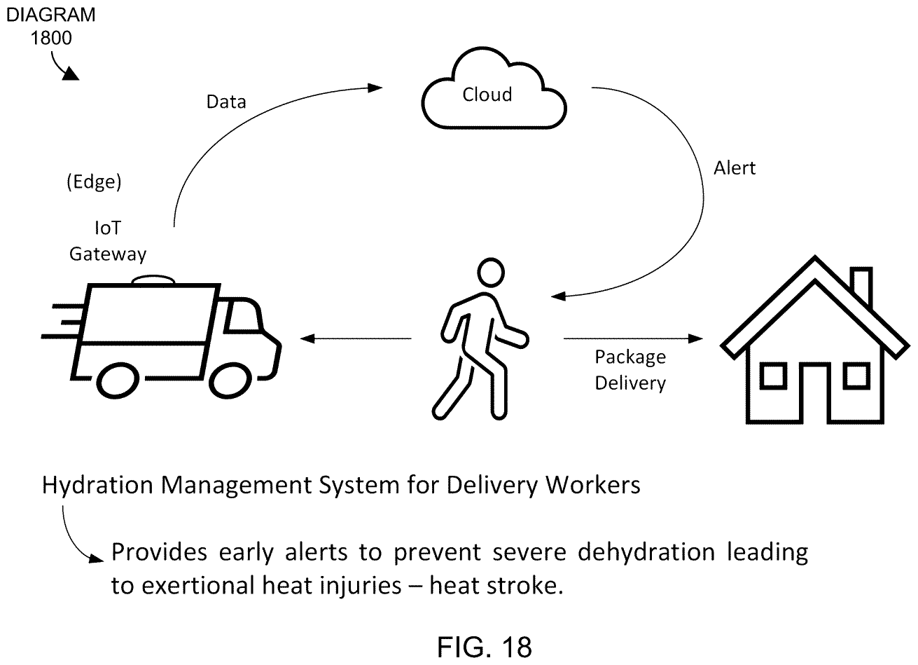

FIG. 18 depicts a diagram 1800 illustrating an example use case of monitoring hydration of delivery and/or mail service workers in accordance with embodiments of the present disclosure. The hydration management system sends early alerts to the designated personnel to prevent severe dehydration leading to exertional heat illness.

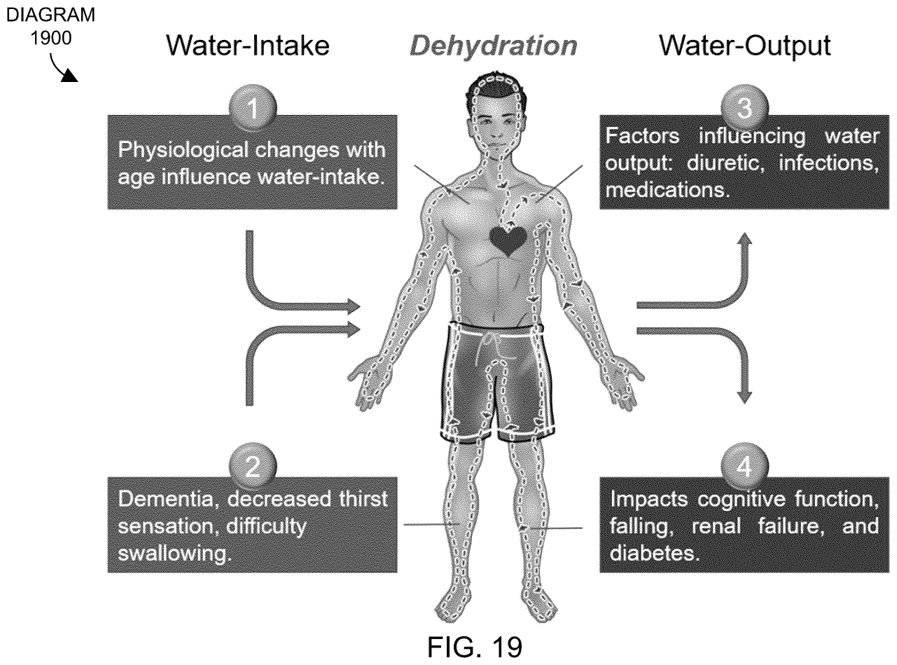

FIG. 19 depicts a diagram 1900 illustrating how dehydration manifests as an imbalance between water-intake and water-output in accordance with embodiments of the present disclosure. Proper hydration maintains the wellness of the body, reduces the risk of illnesses, and improves quality of life for elderly residents by regulating critical body functions including body temperature, blood pressure, and heart rate.

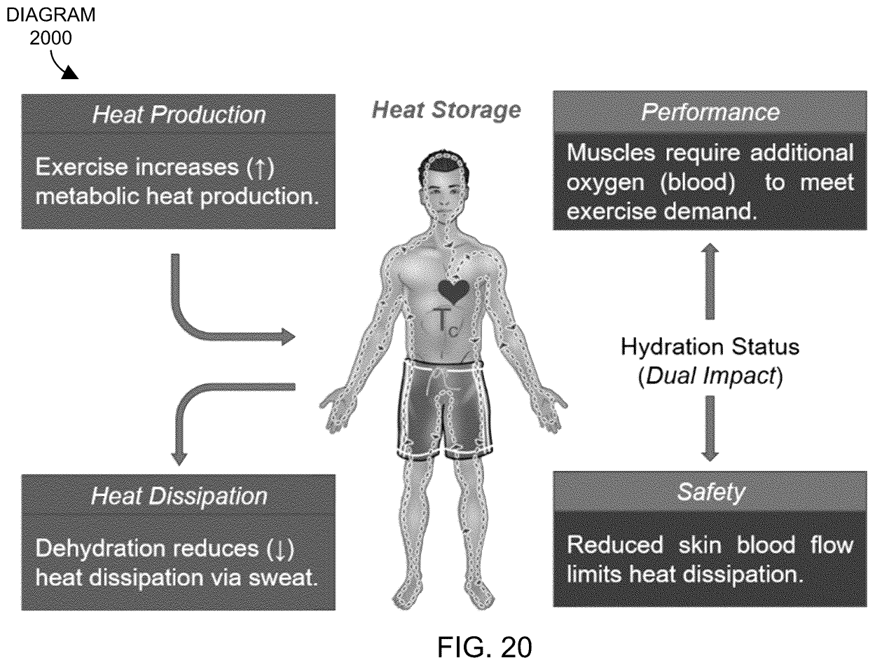

FIG. 20 depicts a diagram 2000 illustrating example of human thermoregulation showing hydration and skin blood flow as two key indicators of how the body dissipates heat during exertional heat stress in accordance with embodiments of the present disclosure. As stated earlier, hydration of the body is an essential physiological parameter to measure but is difficult to measure accurately. Techniques for accessing dehydration include observing a person's appearance (e.g., sunken eyes, cracked lips, etc.), or by examining the volume and color of urine. More quantitative assessment relies on measuring the change in serum osmolality from resident blood samples. Most hydration monitors on the market evaluate the hydration level by measuring physical properties such as conductance, capacitance, and impedance. For reliable readings, the planar electrodes require manual placement against the skin. Shortcomings related to these devices include personal discomfort and large form factors.

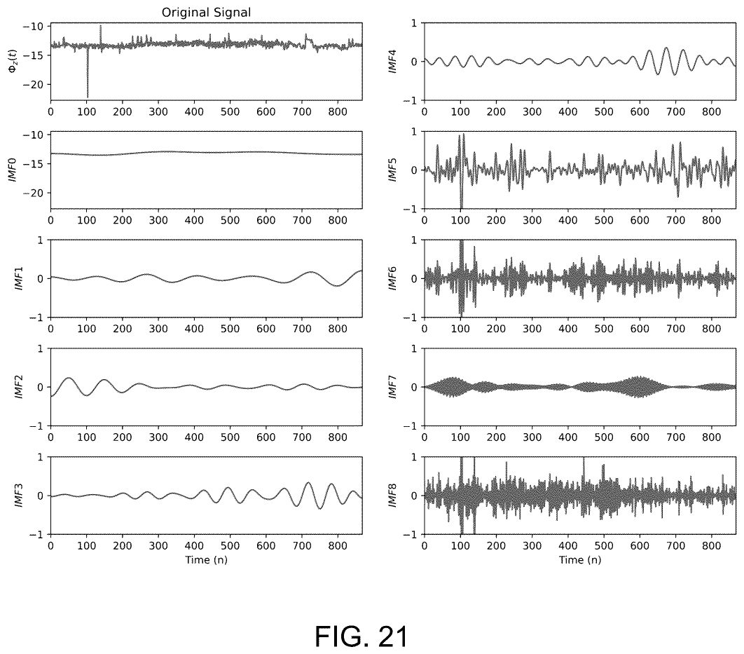

Non-stationary physiological signals contain information across various time scales and frequency content. Individuals naturally induce motion artifacts from complex movements, given their activities related to sports, work, mission, or medical condition. These motion artifacts manifest as parasitic vibrations superimposed on the input data stream. To overcome these artifacts, data-driven signal processing is employed using multi-resolution and multi-scale techniques (e.g., discrete cosine transform, wavelet transform, empirical mode decomposition, empirical wavelet transform, variational mode decomposition) to remove fluctuations and noise artifacts within the raw bioimpedance data stream. In at least one aspect of the present disclosure, the wearable system's Bioimpedance Adaptive Filtering approach employs, for example, empirical wavelet transform to analyze the input signal content. The data-driven transform represents the input signal decomposed into a set of intrinsic mode signals defined by:

$$x ( t ) = {x}_{0} ( t ) + {\sum }_{k = 1}^{N} {x}_{k} ( t )$$

where N denotes the number of modes (e.g., subsignals) with each intrinsic mode signal, x_k (t), behaving as an independent (orthonormal) signal component. Each subsignal captures unique information about the athlete's physical and physiological characteristics. For example, high-frequency components capture signal oscillations related to physical movements such as running, walking, and jogging. FIG. 21 illustrates a representative example of the signal decomposition employing adaptive empirical wavelet method.

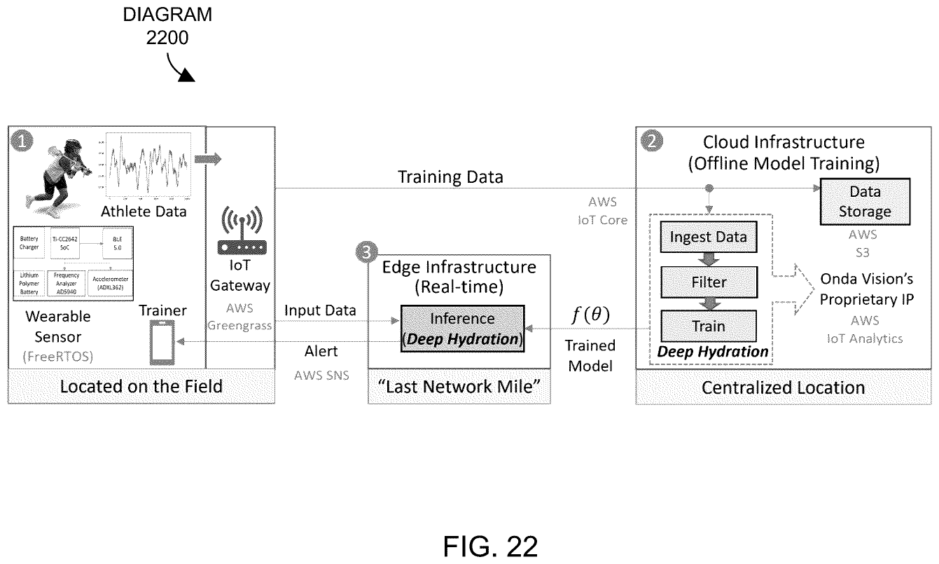

FIG. 22 depicts a diagram 2200 illustrating a wearable hydration monitoring system employing a hybrid edge-cloud architecture to deliver the real-time hydration monitoring to, for example, the athletic staff or medical staff in accordance with embodiments of the present disclosure. FIG. 22 further depicts generating an athlete-specific heat profile. The system is configured to use Amazon Web Services (AWS) IoT components to implement the framework.

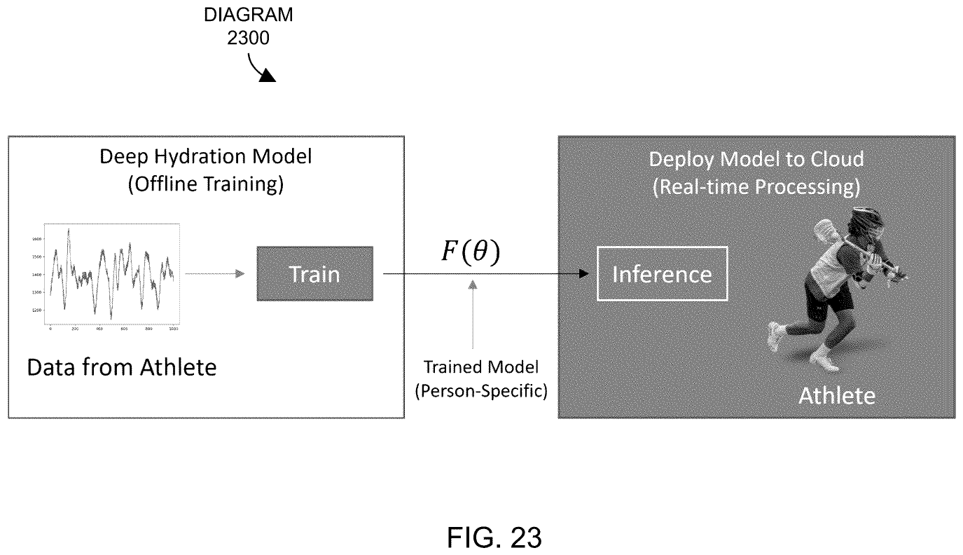

FIG. 23 depicts a diagram 2300 illustrating a continuous stream of data for training and an artificial intelligence (e.g., deep learning) model deployment in accordance with embodiments of the present disclosure. The system generates and tailors a heat profile to the individual's unique physiological characteristics and treats each athlete individually for personalized safety and performance.

The wearable system captures local bioimpedance parameters, including resistance (R), reactance (X_c), phase angle (φ_Z), and the characteristic frequency (f_c). The frequency-dependent phase angle (φ_Z), defined as the arctangent of the Xc divided by R, denotes a key biomarker for hydration assessment of athletes:

$${ϕ}_{Z} ( f ) = - {tan}^{- 1} ( \frac{{X}_{c} ( f )}{R ( f )} )$$

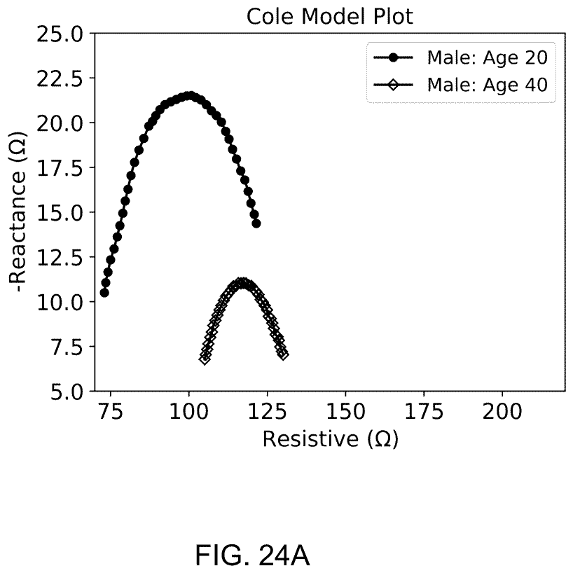

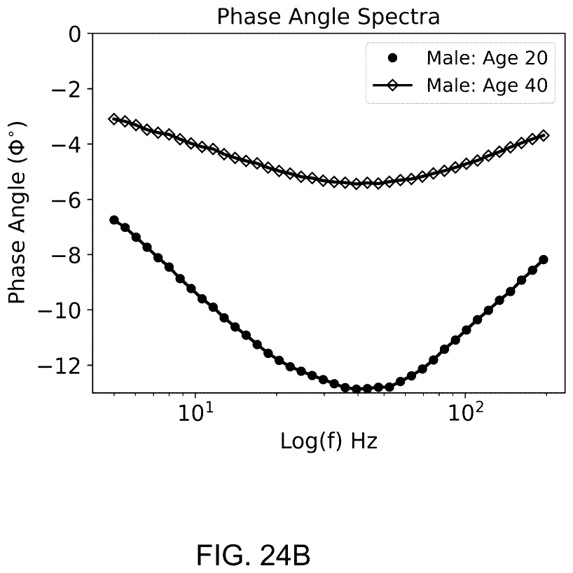

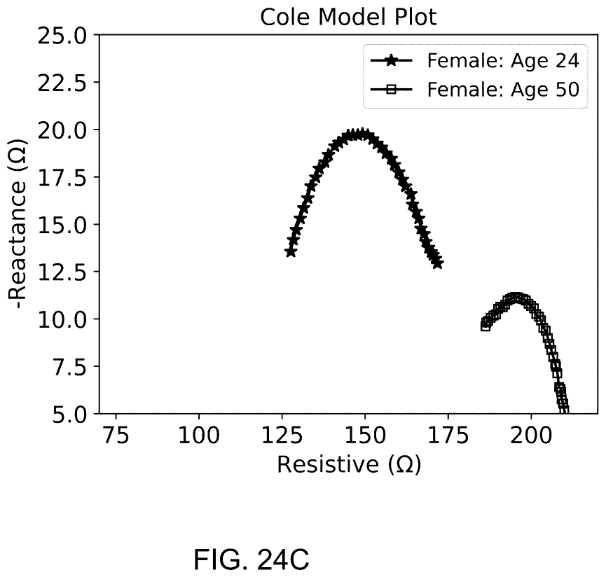

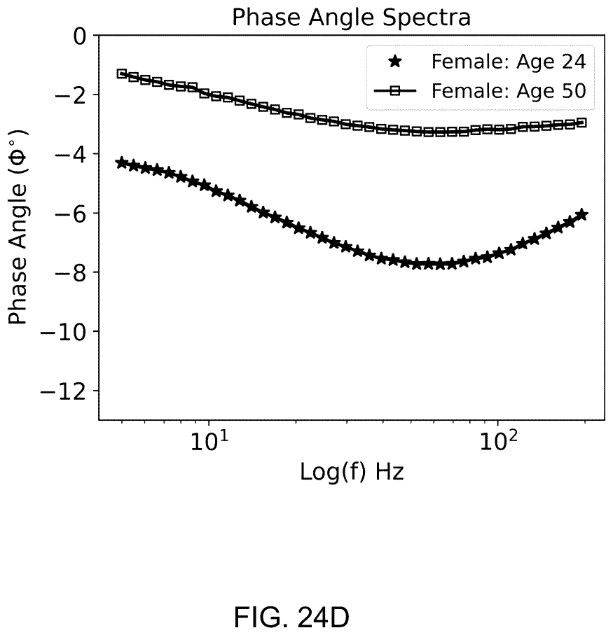

The phase angle examines the cell integrity health (Xc) and the amount of water (R) inside them. The phase angle is also independent of the athlete's height and weight. FIGS. 24A-24D illustrates a representative example of Cole model plots for the wearable bioimpedance measurements collected from healthy male and female subjects across different age ranges: 20-50 years old. FIGS. 24A and 24B illustrate examples of the phase angle measurements for two male subjects (20 and 40 years old) produced by a wearable sensor as disclosed herein, while FIGS. 24C and 24D show the phase angle measurements for two female subjects (24 and 50 years old).

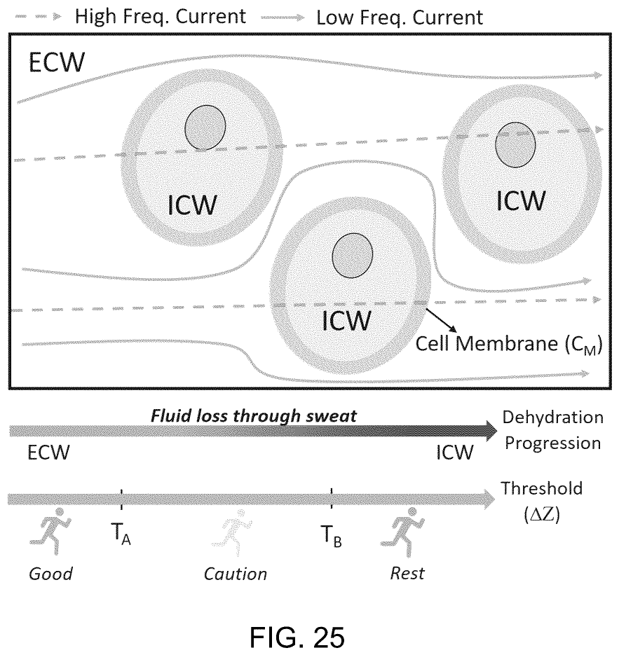

In at least one aspect of the present disclosure, a hydration monitoring system is disclosed. The hydration monitoring system is configured to provide cellular level hydration assessment by measuring extracellular water (ECW) and intracellular water (ICW) fluid compartments. For example, and without limitation, the hydration monitoring system captures a plurality of frequencies to improve the accuracy of the hydration assessment. FIG. 25 illustrates a path of high and low frequency current through human tissue. Fluid loss starts from the ECW compartment and then the ICW compartment as dehydration progresses.

Human physiology indicates during exertion (sweat) fluid exits the body starting with the ECW compartment. Fluid loss signaled by changes in low-frequency bioimpedance measurements (5 kHz-fc) where the range denotes the ECW compartment since the applied current does not penetrate the cell membrane. Fc denotes the characteristic (cross-over) frequency when the current begins to penetrate the cell membrane.

As work intensity and dehydration (water loss) progresses, fluid starts to exit the ICW into the ECW. This fluid movement is induced by osmosis given the higher concentration of sodium (Na+) in the ECW. This is represented by change in high-frequency measurements (fc-195 kHz) since the applied current passes through both the ECW and ICW compartments. These fluid dynamics lead to a notional dehydration thresholding scheme:

$$T ( {Δϕ}_{Z} ) = \{ \begin{matrix}Good , & {Δϕ}_{Z} \leq {T}_{A} \\ Caution , & {T}_{A} < {Δϕ}_{Z} \leq {T}_{B} \\ Rest , & {Δϕ}_{Z} > {T}_{B}\end{matrix}$$

where Δφ_{z} denotes the percent change in phase angle. T_{A} and T_{B} represent the dehydration thresholds. The hydration monitoring system can provide cellular level hydration assessment based by measuring the ECW and ICW fluid compartments. At least two frequencies (e.g., one low and one high) are utilized to capture fluid patterns.

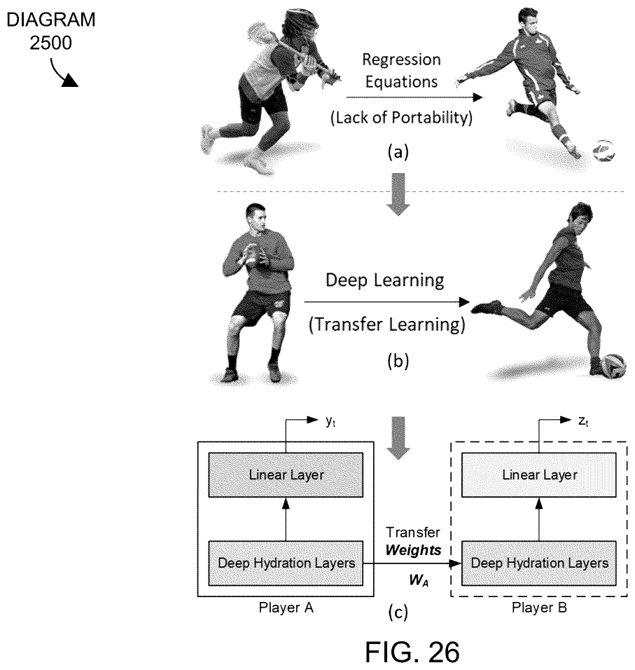

FIG. 26 depicts a diagram 2500 illustrating a wearable hydration monitoring system providing a framework for inter-athlete transfer learning approach for large-scale model (e.g., deep learning) deployment in accordance with embodiments of the present disclosure. The wearable hydration monitoring system offers a novel approach for Inter-Athlete Transfer Learning to support team-based hydration monitoring. The model training approach transfers the lower layer features (weights) from the Temporal Blocks and then learn the higher-level features from the Linear Layers. The transfer learning algorithm randomly initializes the weights of the linear layer. Next, the deep learning model is trained on data from one athlete (Player A) and then the pre-trained model is used to fine-tune the model using the data from a different athlete (Player B). The learning framework speeds up training and prevents training each athlete's deep learning model from scratch.

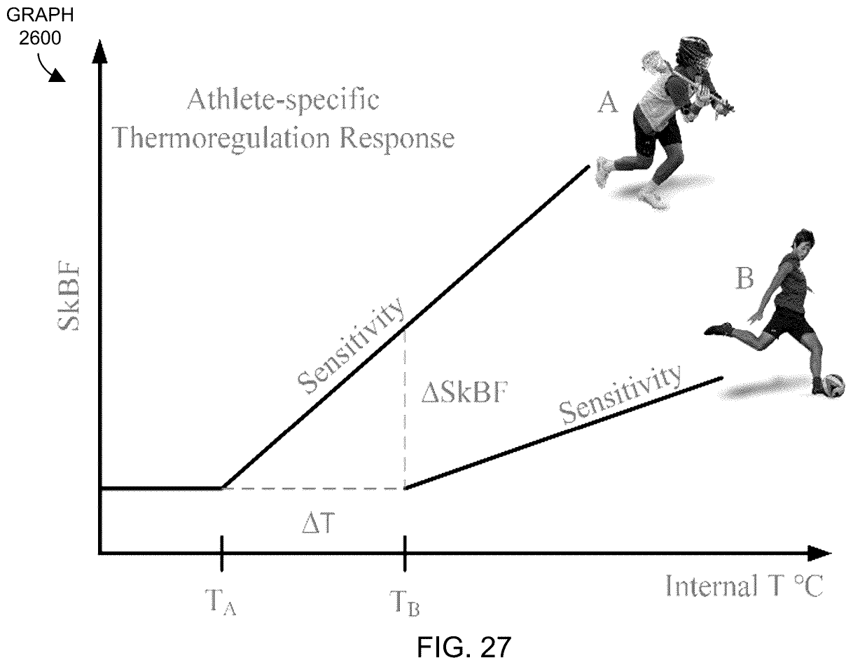

FIG. 27 depicts a graph 2600 illustrating an example of internal temperature versus skin blood flow (SkBF) describing athlete-specific thermoregulation sensitivity to exertional heat stress in accordance with embodiments of the present disclosure. During intense physical activity in the heat, the human thermoregulatory system activates two concurrent mechanisms for heat dissipation. The first mechanism is sweating for evaporative cooling and the second mechanism is skin blood flow (SkBF) for heat transfer. As generally understood in the prior art, the relationship between these two mechanisms can be modeled as a thermoregulation response curve. This nonlinear activation function with thresholds TA (Player A) and TB (Player B) represent the sweating sensitivity (slope of the line) with respect to internal temperature versus skin blood flow. Player A and Player B exhibit different thresholds along with different sensitivities resulting in substantially different responses for heat dissipation. Sensitivity thresholds and responses vary uniquely depending on the athlete's physiological characteristics, heat acclimatization, and fitness levels. Both mechanisms depend on proper hydration status to reduce fatigue and maintain safe levels of core body temperature. A heat profile based on wearable bioimpedance measurements provides new insights into the performance of individuals/persons (e.g., athletes, farmworkers, construction workers, military personnel, elderly, etc.) under heat stress conditions.

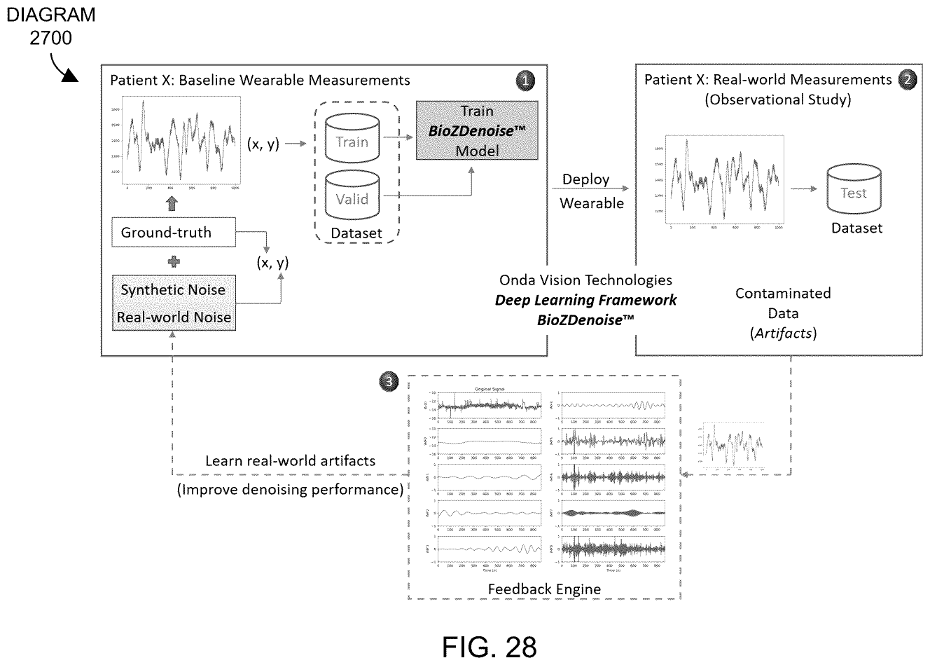

FIG. 28 depicts a diagram 2700 illustrating a framework for bioimpedance denoising using a data-driven feedback engine in accordance with embodiments of the present disclosure. A deep learning framework (i.e. BioZDenoise™) directly integrates knowledge of the real-world into the training procedure to learn complex noise patterns.

The system uses a framework for supervised learning for bioimpedance time-series signal denoising. The framework generates a pair of bioimpedance data (x, y) where x denotes the ground-truth signal (baseline), and y represents the noisy (contaminated) signal. The dataset (x, y) drives the end-to-end training of the system's deep learning model. The model is initialized with synthetic noise, and afterward, the dataset is augmented with real-world noise extracted from the test data collected during the observational study. The system constructs a feedback loop to integrate real-world artifacts directly into the training procedure to improve the denoising performance. Signal processing methods minimize noise artifacts from the raw bioimpedance measurements. The rationale for robust artifact removal supports the need to generate relevant data from real-world scenarios. Data-driven signal processing is employed using multi-resolution or multi-scale techniques (e.g., discrete cosine transform, wavelet transform, empirical mode decomposition, empirical wavelet transform, variational mode decomposition) to drive the feedback engine and extract noise artifacts from the raw bioimpedance time-series data. The system includes artificial intelligence or deep learning architectures that exploit the temporal and spatial information of the bioimpedance time-series data.

The hydration monitoring systems described herein are easily adapted for transferring data and computationally intensive processing tasks to the cloud. With this implementation, the wearable device on-device processing requirements may be reduced. This improves the device's performance and responsiveness and reduces the need for frequent hardware updates. The cloud platform enables near real-time analysis of data collected by the wearable device.

Use cases and implementations of the systems described herein include monitoring hydration of status of construction workers, agricultural workers, athletes, diabetes patients, cancer patients, congestive heart failure (CHF) patients, chronic obstructive pulmonary disease (COPD) patients, kidney patients, dementia patients, elderly, children, infants, ship builders, oil and gas workers, military personnel, delivery drivers and personnel, industrial and factory workers.

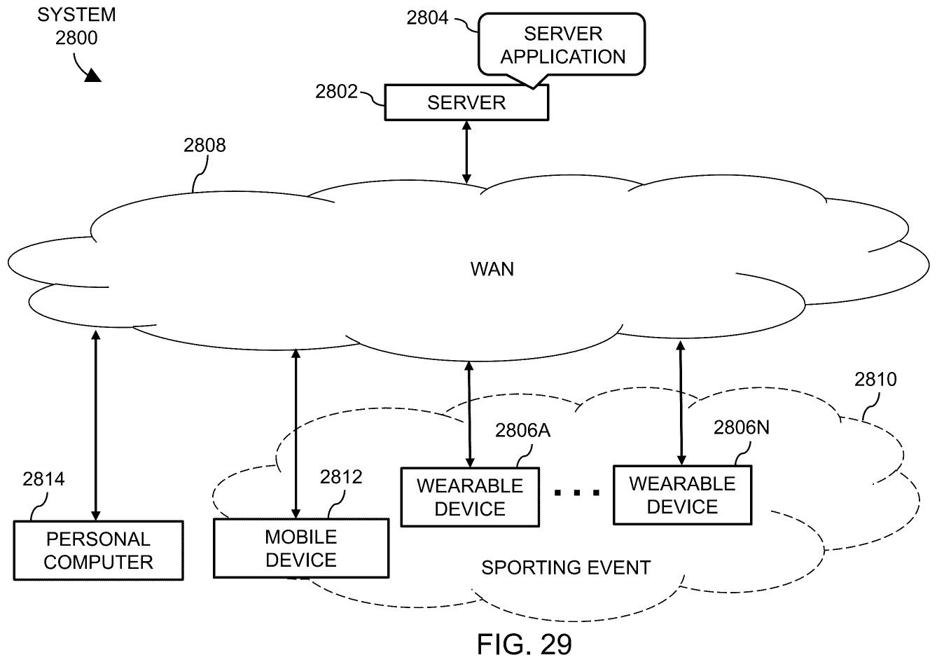

FIG. 29 depicts a block diagram illustrating a system 2800 that includes a server 2802 executing a server application 2804 for monitoring hydration in accordance with embodiments of the present disclosure. The server application 2804 is configured for monitoring a plurality of wearable devices 2806A-2806N over a wide area network (WAN) 2808. The plurality of wearable devices 2806A-2806N are configured for monitoring hydration for a plurality of athletes (not shown in FIG. 29) participating in a sporting event 2810 and transmitting hydration data to the server application 2804 in near real-time. The system 2800 also includes a mobile device 2812 configured to receive analysis data based on the hydration data in near real-time. The analysis data may be in one or more of the dashboard formats of FIGS. 4-6. The mobile device 2812 may also be used to on-board the plurality of wearable devices 2806A-2806N for the sporting event 2810. The mobile device 2812 may be a laptop computer, a smart tablet, a smart phone, a smart watch, smart glasses, an extended reality (XR) headset, a virtual reality (VR) headset, or an augmented reality (AR) headset. The system 2800 also includes a personal computer 2812 that may be used for administering the system 2800 including the server application 2804.

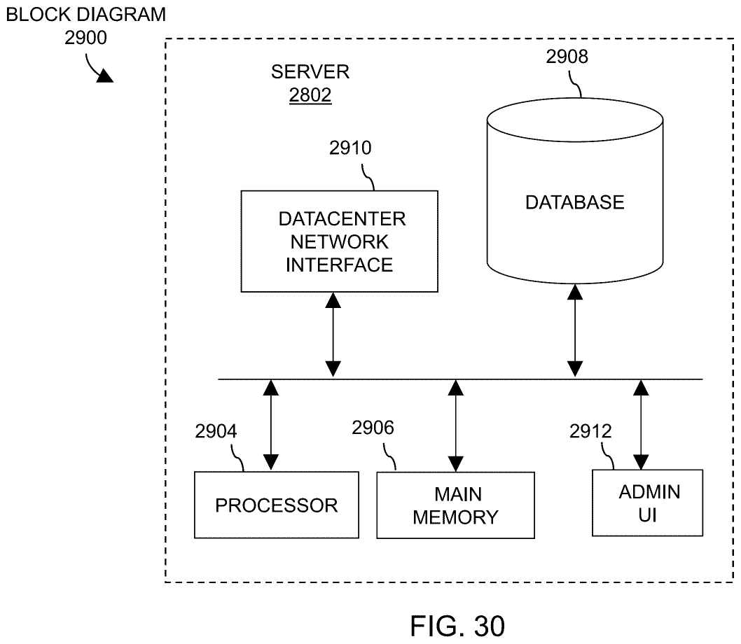

FIG. 30 depicts a block diagram 2900 illustrating one embodiment of the server 2802 of the system 2800 of FIG. 29 in accordance with embodiments of the present disclosure. The server 2802 may be resident in a cloud-based computing environment. The server 2802 may include at least one processor 2904, a main memory 2906, a database 2908, a datacenter network interface 2910, and an administration user interface (UI) 2912. The server 2802 may be configured to host the Ubuntu® server or the like. In some embodiments Ubuntu® server may be distributed over a plurality of hardware servers using hypervisor technology.

The processor 2904 may be a multi-core server class processor suitable for hardware virtualization. The processor may support at least a 64-bit architecture and a single instruction multiple data (SIMD) instruction set. The main memory 2906 may include a combination of volatile memory (e.g., random access memory) and non-volatile memory (e.g., flash memory). The database 2908 may include one or more hard drives.

The datacenter network interface 2910 may provide one or more high-speed communication ports to the data center switches, routers, and/or network storage appliances (and ultimately the WAN 2808 of FIG. 29). The datacenter network interface 2910 may include high-speed optical Ethernet, InfiniBand (IB), Internet Small Computer System Interface (ISCSI), and/or Fibre Channel interfaces. The administration UI 2912 may support local and/or remote configuration of the server 2802 by a datacenter administrator. In some embodiments the administration UI 2912 may be communicatively coupled directly with the personal computer 2814 of FIG. 29 for management of the server application 2804 of FIG. 29.

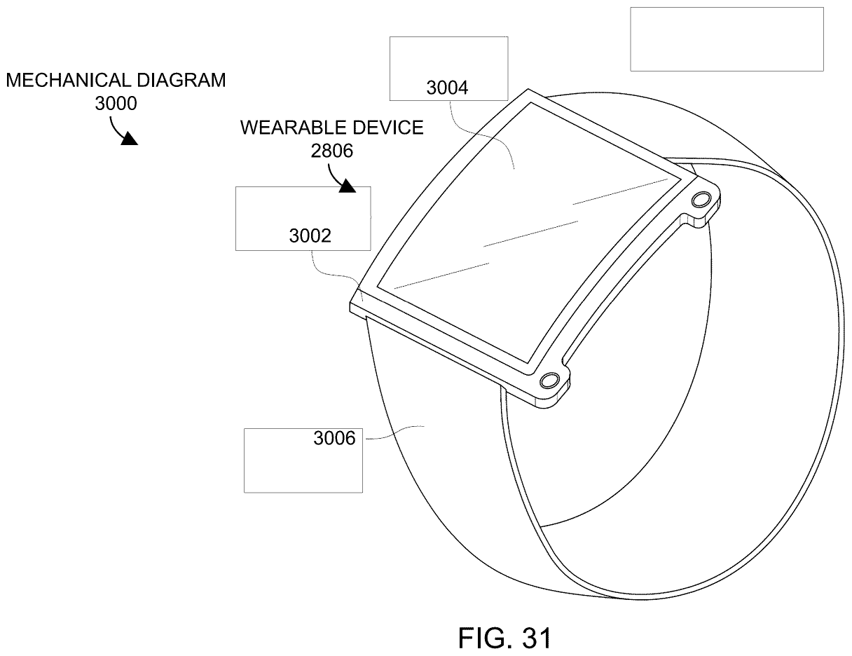

FIG. 31 depicts a mechanical diagram illustrating one embodiment of the wearable device 2806 of the system 2800 of FIG. 29 in accordance with embodiments of the present disclosure. The wearable device 2806 includes an electronic enclosure 3002 with a display 2904. The wearable device 2906 also includes a wrist band 2906. The wearable device may be configured to wirelessly communicate with additional sensors positioned on the athlete.

In at least one embodiment of the present disclosure, the hydration monitoring system includes a full-arm sleeve with at least one compartment (e.g., pocket) for receiving the hydration wearable device. The full-arm sleeve is configured to position the hydration wearable device at a designated area of interest on a user. For example, and without limitation, the designated area of interest may include the radial artery pulse-point at a wrist, a brachial artery pulse-point at the bicep, a pulse-point of the ulnar artery, a popliteal artery pulse-point, and a tibial artery pulse-points. The full-arm sleeve may include a compressive strap that enables adjustable attachment to the patient. Advantageously, the strap helps maintain the positioning of the wearable device while a user is moving, thereby maintaining contact between the electrodes of the wearable device and a user's skin.

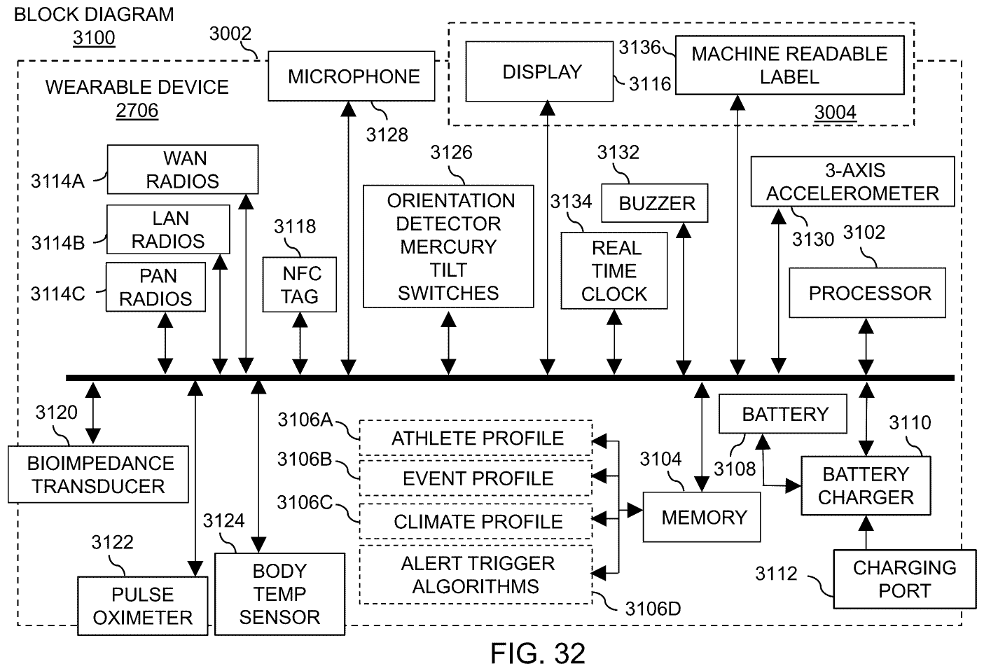

FIG. 32 depicts a block diagram 3100 illustrating one embodiment of the wearable device 2806 of the system 2800 of FIG. 29. The wearable device 2806 includes a processor 3102 and a memory 3104 in accordance with embodiments of the present disclosure. In some embodiments, the memory 3104 or a portion of the memory 3104 may be integrated with the processor 3102. The memory 3104 may include a combination of volatile memory and non-volatile memory. In some embodiments the processor 3102 and the memory 3104 may be embedded in a microcontroller. The processor 3102 may be the Snapdragon® 4100 processor, the NXP Kinetix® microcontroller unit (MCU), or the like. The memory 3104 may be configured to receive an athlete profile 3106A and an event profile 3106B of the individual to me monitored. The memory may also be configured to receive a climate profile 3106C and one or more alert trigger algorithms 3106D. The alert trigger algorithms 3106D may use the athlete profile 3106A, the event profile 3106B, and/or climate profile 3106C to directly trigger a dehydration alert to the athlete. In other embodiments, the alert trigger algorithms 3106D, the athlete profile 3106A, the event profile 3106B, and/or climate profile 3106C may be implemented within the server 2802 of FIG. 29.

The wearable device 2806 also includes a battery 3108, a battery charger 3110, and a charging port 3112. The charging port 3112 may be a wireless charging port.

A plurality of radios for interfacing to multiple wireless networks is also implemented in the wearable device 2806. The wide area network (WAN) radios 3114A may include 2G, 3G, 4G, and/or 5G technologies. The local area network (LAN) radios 3114B may include Wi-Fi technologies such as 802.11a, 802.11b/g/n, 802.11ac, 802.11.ax or the like circuitry. The PAN radios 3114C may include Bluetooth® technologies. In other embodiments, the wearable device 2806 may include a wide-band direct sequence spread spectrum (WBDSSS) wireless transceiver (not shown in FIG. 32). The WBDSSS wireless transceiver may be configured to transmit and receive signals within a 902 megahertz (MHz) to 928 MHz frequency band to a specialized access point (not shown in FIG. 32) positioned at the sporting event 2810.

A display 3116 is included on the faceplate 3004 of the enclosure 3002 and under control of the processor 3102. The wearable device 2806 also includes a near field communication (NFC) tag 3118 for secure identification to one or more NFC readers. The NFC tag 3118 may be read by the mobile device 2812 for onboarding the wearable device 2806 for the sporting event 2810. In further embodiments, the wearable device 2806 may include an NFC reader (not shown in FIG. 29) under control of the processor 3102.

The wearable device 2806 includes a bioimpedance transducer 3120 configured for detecting hydration of the individual being monitored. In certain embodiments, the bioimpedance transducer 3120 may include multiple components positioned at different locations on the athlete. The multiple components may be wirelessly coupled via the PAN radios 3114C. The bioimpedance transducer 3120 uses a small electrical current to determine an impedance on body tissues passed through. Using this impedance with different models, water content within the tissues may be estimated. This analysis may be performed at the wearable device 2806 and/or within the server application 2804 of FIG. 29. Additional data such as body temperature, pulse rate, oxygen levels, skin temperature, and activity levels may be used in determining the hydration data.

The wearable device 2806 includes a pulse oximeter 3122 and a body temperature sensor 3124 for monitoring multiple vital signs of the individual including pulse rate, blood oxygen levels, and skin temperatures. The wearable device 2806 also includes an orientation detector 3126 including mercury tilt switches, a microphone 3128, and a three-axis accelerometer 3130. The orientation detector 3126 is configured to detect a relative position to gravity of the wearable device 2806. The three-axis accelerometer 3130 is configured to detect instantaneous movements on x, y, and z-axis of the wearable device 2806. The microphone 3128 may be used to detect voice communication from the individual and/or ambient sounds.

The wearable device 2806 includes a buzzer 3132 and/or vibration capability under control of the processor 3102 and configured to gain the attention of the athlete being monitored. The wearable device 2806 also includes a real time clock 3134 for timestamping any monitored data and possibly changing alert trigger algorithms based on time-of-day and/or day-of-week. A machine readable label 3136 is also implemented within and/or on the faceplate 3004 of the enclosure 3002. The machine readable label 3136 may include a barcode, a quick response (QR) code, a radio-frequency identification (RFID) tag, or the like. In some embodiments, the RFID tag may be a smart RFID under control of the processor 3102. The machine readable label 3136 may be used by the mobile device 2812 for provisioning the wearable device 2806 when on-boarding or off-boarding an individual under monitoring. In some embodiments, the machine readable label 3136 may be replaced by using a BLE advertisement packet and/or the NFC tag 3118 may be used for provisioning the wearable device 2806.

In some embodiments, the wearable device 2806 may include global navigation satellite system (GNSS) radios 3218 or the like (not shown in FIG. 32) readable by the processor 3102 to identify a location of the wearable device 2706. Also (not shown in FIG. 32), a plurality of analog-to-digital converters (ADCs) and digital-to-analog converters (DACs) may be used to interface the various components/sensors to the processor 3102. In certain embodiments, the wearable device 2806 may also include a speaker and associated amplifier circuitry (not shown in FIG. 32) for communicating directly to an individual being monitored.

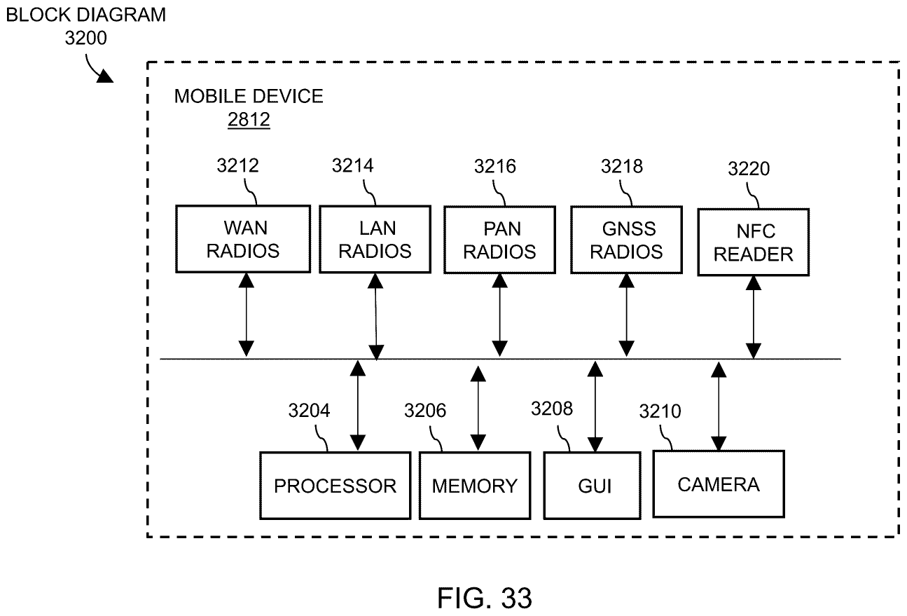

FIG. 33 depicts a block diagram 3200 illustrating one embodiment of the mobile device 2812 in accordance with embodiments of the present disclosure. The mobile device 2812 may be a smart phone, a smart tablet, or the like. The mobile device 2812 includes at least one processor 3204, a memory 3206, a graphical user interface (GUI) 3208, a camera 3210, wide area network (WAN) radios 3212, local area network (LAN) radios 3214, and personal area network (PAN) radios 3216.

In some embodiments, the processor 3204 may be a mobile processor such as the Qualcomm® Snapdragon™ mobile processor. The memory 3206 may include a combination of volatile memory (e.g., random access memory) and non-volatile memory (e.g., flash memory). The memory 3206 may be partially integrated with the processor 3204. The GUI 3208 may be a touchpad display. The WAN radios 3212 may include 2G, 3G, 4G, and/or 5G technologies. The LAN radios 3214 may include Wi-Fi technologies such as 802.11a, 802.11b/g/n, 802.11ac, 802.11.ax or the like circuitry. The PAN radios 3216 may include Bluetooth® technologies.

The mobile device 2812 also includes GNSS radios 3218 for determining current location data of the mobile device 2812. The camera 3210 may be used to capture a barcode and/or quick response (QR) code as needed for onboarding of the wearable device 2806. The mobile device also includes an NFC reader 3220 configured for reading the NFC tag 3118 of the wearable device 2806 of FIG. 32 for the onboarding operation.

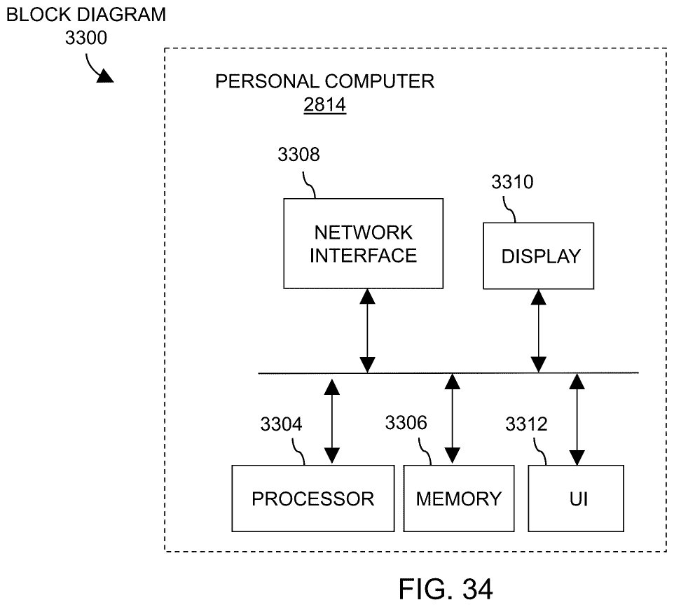

FIG. 34 depicts a block diagram 3300 illustrating one embodiment of the personal computer 2814 of the system 2800 of FIG. 29 in accordance with embodiments of the present disclosure. The personal computer 2814 includes at least one processor 3304, a memory 3306, a network interface 3308, a display 3310, and a UI 3312. The memory 3306 may be partially integrated with the processor 3304. The UI 3312 may include a keyboard and a mouse.

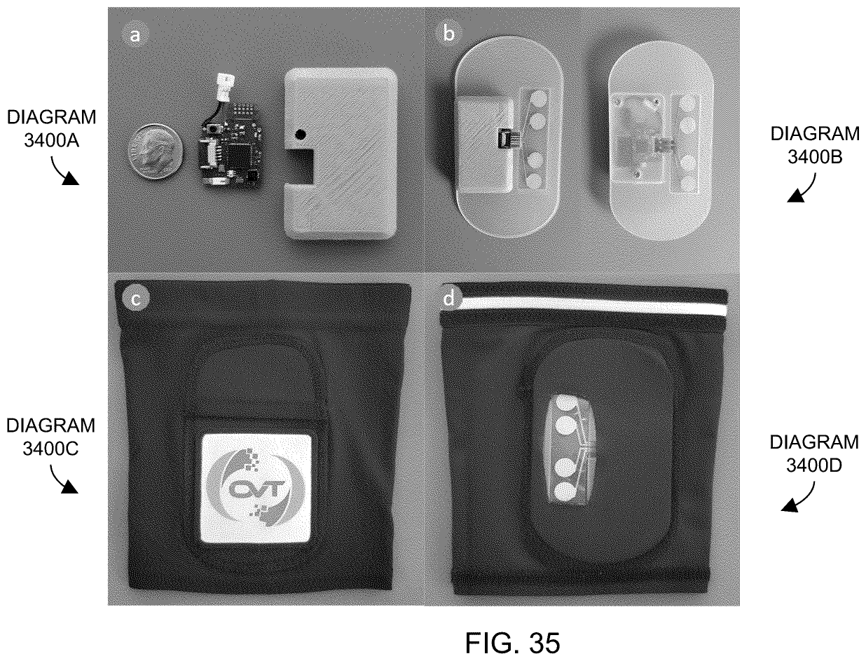

FIG. 35 depicts diagrams 3400A-3400D illustrating another embodiment of the wearable device 2806 of the system 2800 of FIG. 29 that may be used with different flexible form factors suitable for specific locations (upper arm, forearm, calf, etc.) on a user (e.g., athlete, etc.). In diagram 3400A, a miniaturized printed circuit board (PCB) assembly and a protective housing are depicted with a dime to show relative size. The PCB assembly includes a Texas Instruments System-on-a-chip (SoC) (TI-CC2642) equipped with Bluetooth Low Energy (BLE 5.0) interfacing with an impedance measuring analog front end (AFE) (AD5941, Analog Devices, Inc.).

In diagram 3400B, the PCB assembly and protective housing are depicted integrated with a four-electrode assembly and configured for four-electrode bioimpedance measurements of the user using a tetra-polar pattern. Each electrode has a size of approximately 10 millimeters (mm)×10 mm. The two outer electrodes are configured to inject a small current into the local body region while the two inner electrodes (spaced approximately 10 mm apart) measure the voltage. The wearable device conforms to IEC 6061 Safety Protocols to limit alternating current (AC) and direct current (DC) injected into the body of the user. The PCB assembly further includes a three-axis accelerometer (ADXL362, Analog Devices, Inc.) for recording activity levels and motion artifacts. Additionally, the PCB assembly includes a 3.7 volt 150 milliampere-hour lithium polymer battery regulated to approximately 3.3 volts by a linear voltage regulator (ADP160, Analog Devices, Inc.). Overall, the wearable device measures approximately 20 mm by 26 mm by 7.4 mm. Additionally, the wearable device has a mass of approximately 12.1 grams.

In diagram 3400C, an outside of a wearable armband is depicted for securing the wearable device to the user. The wearable armband offers advantages including long-term adhesion to the skin, reuse, and a flexible form factor.

In diagram 3400D, an inside of the wearable armband is depicted with the wearable device installed in a sleeve. The four sensor electrodes (two inner electrodes and two outer electrodes) are exposed through an inner slit of the wearable armband.

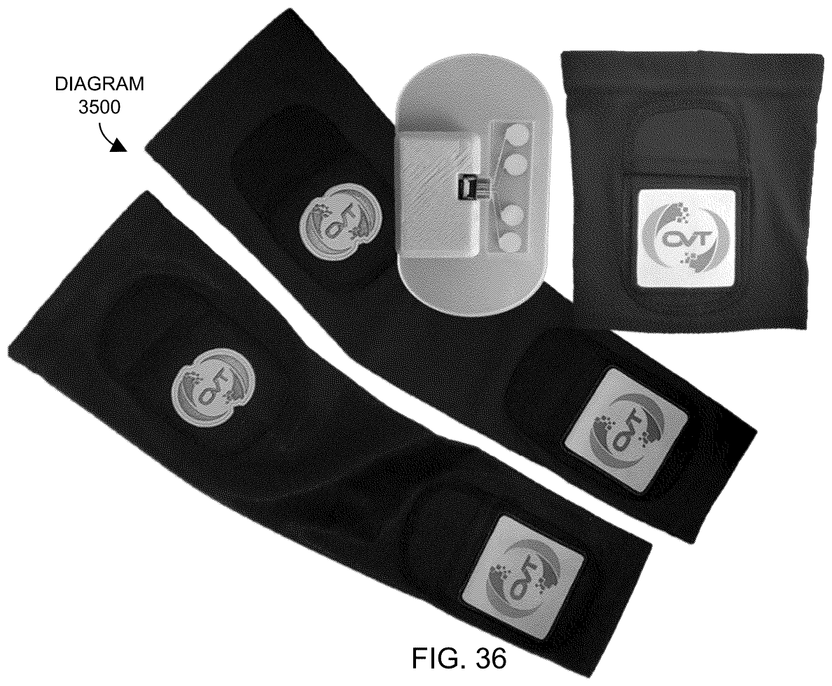

FIG. 36 depicts a diagram 3500 illustrating the wearable device and wearable armband of FIG. 35 in accordance with embodiments of the present disclosure. Additionally, diagram 3500 depicts two additional flexible form factors for use with the wearable device. The wearable armband and the two additional flexible form factors are configured to allow collecting bioimpedance measurements from the upper biceps, a forearm, or a calf as needed by a user.

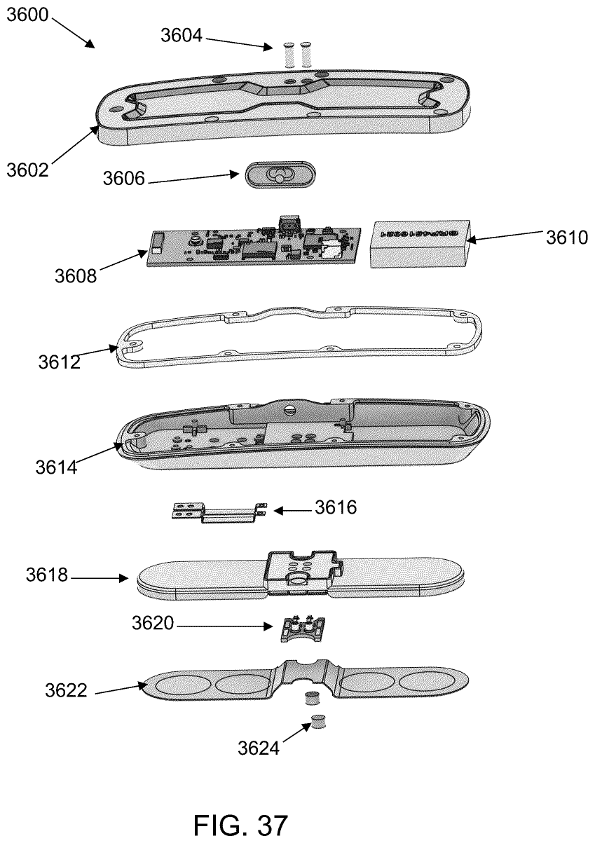

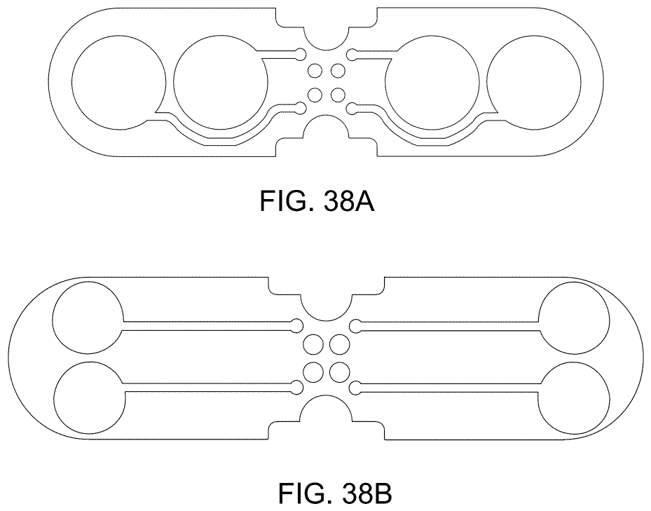

FIG. 37 illustrates an exploded view of a hydration monitoring wearable device 3600 according to at least one embodiment of the present disclosure. The hydration monitoring wearable device 3600 includes a wearable housing cover 3602, a plurality of LED light pipes 3604, a button 3606, controllable electronics 3608, a power supply 3610 (e.g., rechargeable battery), a housing gasket 3612, a wearable housing base 3614, a plurality of charging strips 3616, a sensor strip 3618, a pogo pin PC 3620, a plurality of sensor electrodes 3622, and a plurality of magnets 3624. FIGS. 38A and 38B illustrate electrode configurations according to at least two aspects of the present disclosure. The hydration monitoring wearable device 3600 is configured for the capture of bioimpedance data from a subject when the electrodes are in contact with the subject's skin. The hydration monitoring wearable device is further configured for continuous real-time monitoring of the subject's hydration status. Advantageously, the hydration monitoring wearable device is configured to generate an alert or notification when a subject's hydration status is excess a predetermined threshold. The hydration monitoring wearable device is further configured to update a subject's hydration profile based on the subject's activity.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: Ruby, JavaScript, Java, Python, Ruby, PHP, C, C++, C#, Objective-C, Go, Scala, Swift, Kotlin, OCaml, or the like. The program code may execute entirely on the user's computer, partly on the user's computer, as a stand-alone software package, partly on the user's computer, and partly on a remote computer or entirely on the remote computer or server.

Aspects of the present invention are described in the instant specification with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a user” can include a plurality of such users, and so forth. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A programmatic method for monitoring hydration, the programmatic method comprising:
receiving first continuous health data including first hydration data associated with a first subject from a first portable health monitoring device;
determining first analysis data based on the first continuous health data; and
providing a portion of the first analysis data to a display, wherein:
the first analysis data includes a first alert; and
the first alert is a first notification identifying a hydration status of the first subject.

2. The programmatic method of claim 1, wherein the first hydration data is based on bioimpedance monitoring of the first subject using the first portable health monitoring device.

3. The programmatic method of claim 1, wherein the first hydration data includes first water content data.

4. The programmatic method of claim 3, wherein the first hydration data includes first electrolyte data, wherein the first electrolyte data includes at least one of calcium data, chloride data, magnesium data, phosphorus data, potassium data, and sodium data.

5. The programmatic method of claim 2, wherein the bioimpedance monitoring includes magnitude and/or phase angle.

6. The programmatic method of claim 1, wherein the first analysis data includes a first recommended treatment based on the hydration status of the first subject.

7. The programmatic method of claim 6, wherein the first recommended treatment includes a first recommended electrolyte intake, wherein the first recommended electrolyte intake includes at least one of calcium, chloride, magnesium, phosphorus, potassium, and sodium.

8. The programmatic method of claim 1, wherein determining the first analysis data is further based on a machine learning model, wherein the machine learning model is based on additional hydration data of additional subjects.

9. The programmatic method of claim 8, wherein the machine learning model is configured for identifying temporal patterns of a bioimpedance profile associated with the first subject, wherein based on the identified temporal patterns of the bioimpedance profile, the machine learning model is further configured to update the hydration status of the first subject.

10. The programmatic method of claim 1, wherein the first portable health monitoring device is a first wearable health monitoring device, wherein the first wearable health monitoring device is configured to make continuous bioimpedance measurements on at least one of a wrist, an upper arm, a forearm, or a calf of the first subject.

11. The programmatic method of claim 1, wherein determining the first analysis data is further based on data-driven signal processing is using at least one of a discrete cosine transform, a wavelet transform, an empirical mode decomposition, an empirical wavelet transform, and a variational mode decomposition.

12. A system for monitoring hydration, the system comprising
at least one wearable health monitoring device associated with at least one subject; and
a computing device wirelessly coupled with the at least one wearable device, wherein the computing device is configured for:
receiving first health data including first hydration data from the at least one wearable health monitoring device;
determining a hydration status of the at least one subject based on the first health data; and
providing a portion of the first analysis data to a display, wherein:
the hydration status includes a first alert; and
the first alert is a first notification for at least one action based on the hydration status.

13. The system for monitoring hydration of claim 12, wherein the at least one wearable health monitoring device is configured to capture bioimpedance data corresponding to the at least one subject.

14. The system for monitoring hydration of claim 13, wherein the bioimpedance data includes at least one of resistance, reactance, phase angle, magnitude, or characteristic frequency.

15. The system of monitoring hydration of claim 13, wherein the at least one wearable health monitoring device includes a plurality of electrodes positioned on a surface of the at least one wearable health monitoring device, wherein the at least one wearable health monitoring device is configured to capture the bioimpedance data when the plurality of electrodes is in contact with the at least one subject.

16. The system of monitoring hydration of claim 15, wherein the bioimpedance is based on voltage or current between at least two of the electrodes of the plurality of electrodes.

17. The system of monitoring hydration of claim 13, wherein the at least one wearable health monitoring device is configured to capture the bioimpedance data while positioned on at least one of a wrist, an upper arm, a forearm, or a calf of the first subject.

18. A system for monitoring hydration, the system comprising
a plurality of wearable health monitoring devices associated with a plurality of subjects; and
a computing device wirelessly coupled with the plurality of wearable health monitoring devices, wherein the computing device is configured for:
receiving first health data including first hydration data from the plurality of wearable health monitoring devices;
determining a hydration status of the plurality of subjects based on the first health data; and
providing a portion of the first analysis data to a display, wherein:
the hydration status includes a first alert; and
the first alert is a first notification for at least one recommended action based on the hydration status.

19. The system for monitoring hydration of claim 18, wherein each wearable health monitoring device of the plurality of wearable health monitoring devices is configured to capture real-time or near real-time bioimpedance data corresponding to at least one subject of the plurality of subjects.

20. The system for monitoring hydration of claim 19, wherein the bioimpedance data includes at least one of resistance, reactance, phase angle, magnitude, or characteristic frequency.

21. The system of monitoring hydration of claim 19, wherein the at least one wearable health monitoring device includes a plurality of electrodes positioned on a surface of the at least one wearable health monitoring device, wherein the at least one wearable health monitoring device is configured to capture the bioimpedance data when the plurality of electrodes is in contact with a corresponding subject.

22. The system of monitoring hydration of claim 21, wherein the bioimpedance is based on voltage or current between at least two of the electrodes of the plurality of electrodes.

23. The system of monitoring hydration of claim 18, wherein the computing device is further configured to generate a hydration profile for each subject of the plurality of subjects based on the health data, wherein the hydration profile includes fluid data corresponding to each subject during dehydration.

24. The system of monitoring hydration of claim 19, wherein the first health data includes extracellular water data and intracellular water data, wherein determining the hydration status of the plurality of subjects based on the first health data includes identifying a change in bioimpedance phase angle, magnitude, resistance, or reactance.
